(19) United States
(12) Patent Application Publication
(10) Pub. No.: US 2021/0100941 A1
(43) Pub. Date: Apr. 8, 2021

(54) COMPOSITIONS AND METHODS FOR REGENERATING CARRIER PROTEIN-CONTAINING MULTIPLE PASS ALBUMIN DIALYSIS FLUID

(71) Applicant: ADVITOS GMBH, München (DE)

(72) Inventors: Bernhard KREYMANN, München (DE); Christoph HÜSSTEGE, München (DE)

(73) Assignee: ADVITOS GMBH, München (DE)

(21) Appl. No.: 16/498,321

(22) PCT Filed: Mar. 28, 2017
(86) PCT No.: PCT/EP2017/057342

§ 371 Date: Sep. 26, 2019

(30) Foreign Application Priority Data
Mar. 28, 2017 (EP) PCTEP2017057342

(51) Int. Cl. A61M 1/16; A61K 31/7004; A61K 33/08; A61K 33/10; A61K 33/14; A61K 33/42; A61K 47/12

(52) U.S. Cl. CPC A61M 1/1676; A61K 31/7004; A61K 33/08; A61K 47/12; A61K 33/14; A61K 33/42; A61K 33/10

(57) ABSTRACT

The present invention provides compositions which can be used to treat a carrier protein-containing multiple pass dialysis fluid in particular in order to ensure regeneration of a carrier protein such as albumin in the dialysis fluid. The invention further relates to kits comprising such compositions and uses thereof as well as to methods for providing and regenerating a carrier protein-containing multiple pass dialysis fluid.

The present invention relates to the field of regeneration of a carrier protein-containing multiple pass dialysis fluid. More specifically, the present invention relates to compositions which can be used to treat a carrier protein-containing multiple pass dialysis fluid in particular in order to ensure regeneration of a carrier protein such as albumin in the dialysis fluid. The invention further relates to methods for providing and regenerating a carrier protein-containing multiple pass dialysis fluid.

When liver or kidney of a human being fail to perform their normal functions, inability to remove or metabolise certain substances results in their accumulation in the body. These substances can be differentiated according to their solubility in water in water-soluble and water-insoluble (or protein-bound) substances. Different extracorporeal procedures are available to help to replace the failing functions. Haemodialysis is the gold standard for treating patients with renal failure. For this purpose a dialyzer is used, which is divided into two compartments by a semipermeable membrane. Blood is passed through the dialyzer's blood compartment, which is separated by the semipermeable membrane from dialysis fluid which passes through the dialysis compartment of said dialyzer. A physiological dialysis fluid should comprise the desired electrolytes, nutrients and buffers in concentrations so that their levels in the plasma are brought to normal.

The routine haemodialysis is of little help for patients with liver failure, especially when they have no accompanying renal failure. This is mainly due to the fact that the main toxins such as metabolites, e.g. bilirubin and bile acids, accumulating in hepatic failure are protein-bound and are therefore hardly removed by conventional (renal) haemodialysis.

In order to improve the efficiency of the dialysis procedure, transport of substances from and to blood is enhanced by the physical phenomenon of convention. This is achieved through dilution of blood before (predilution) and/or after the dialyzer (postdilution). In this way, different substances (harmful and useful) present in the blood of a liver failure patient are removed from blood with the use of a pressure gradient. However, the removal of so-called middle molecules is highly dependent on the filtration volumes.

In order to enhance the removal of the protein-bound substances, the dialysis fluids were modified to comprise a carrier protein such as albumin, which binds to the unbound toxins travelling from blood to the dialysis fluid across the semipermeable membrane. The presence of a carrier protein such as albumin in the dialysis fluid facilitates the removal of protein-bound substances from blood. In particular, albumin is the main carrier protein for protein-bound toxins in the blood. Such a mode of treatment wherein albumin is used to remove protein-bound substances from blood is then called “albumin dialysis”.

A simple method of albumin dialysis, wherein standard renal replacement therapy machines can be used, is “single pass albumin dialysis” (SPAD). In SPAD, the patient's blood flows through a circuit with a high-flux hollow fiber hemodiafilter. The other side of this membrane is cleansed with a carrier protein solution in counter-directional flow, which is discarded after passing the filter.

However, commercially available albumin is very expensive. Therefore, in SPAD high costs incur due to the albumin, which is discarded after a single pass. Therefore, multiple-pass albumin dialysis devices were developed. One such device is the “Molecular adsorbents recirculation system” (MARS), which is an extracorporeal hemodialysis system composed of three different circuits: blood, albumin and low-flux dialysis. Blood is dialyzed against an albumin dialysate. The albumin dialysate is then regenerated in a loop in the MARS circuit by passing through the fibers of the low-flux diaFLUX filter, to clear water-soluble toxins and provide electrolyte/acid-base balance, by a standard dialysis fluid. Next, the albumin dialysate passes through two different adsorption columns to remove protein-bound substances and anionic substances. However, the costs for a treatment with MARS are still very high, in particular due to an expensive “MARS treatment kit”. Moreover, the detoxification efficiency is unsatisfactory: on average only up to 30% reduction of the bilirubin level as a marker for protein-bound substances can be achieved. Although the albumin-based dialysis processes bring about an improvement in the symptoms of hepatic encephalopathy, a normalization of the values cannot be achieved as a consequence of the limited detoxification efficacy and high treatment costs.

WO 03/094998, US 2005/0082225 A1 and WO 2009/071103 A1 describe multiple-pass albumin dialysis wherein the albumin is regenerated by means of modifying the pH, in particular by addition of an acid and a base in order to treat the albumin-containing multiple-pass dialysis fluid. Accordingly, the pH of the fluid is lowered or to increased, thereby reducing the binding of certain toxins to the carrier proteins in the acidic range or in the alkaline range and hence “releasing” the protein-bound toxins in the dialysis fluid from the proteins and increasing the concentration of free toxins in the fluid. The toxins can then easily be removed by filtration. The “free” carrier protein can then enter a further cycle of dialysis.

Such a modification of the pH value of the dialysis fluid enables a dialysis system, wherein the pH value of the dialysis fluid can be adjusted according to the needs of the dialysis procedure. Thereby, (i) a variety of dialysis procedures, e.g. for renal support, liver support and/or lung support (e.g. for treating acidosis) and (ii) multiple organ support, can be realized in one and the same dialysis system.

In view of the above, it is the object of the present invention to provide compositions for treating a carrier protein-containing multiple pass dialysis fluid, which (i) enable the “cleaning” of a carrier protein carrying a toxin by modification of the pH value, (ii) provide the required electrolytes and/or nutrients and (iii) enable an adjustment of the pH of the dialysis fluid (as used for dialysis, i.e. in the dialyzer) to values from 6.35 to 11.4, in particular from 6.5 to 10, preferably from 7.4 to 9. Thus, such compositions can be used in different dialysis procedures, e.g. for renal support, liver support, lung support; for multiple organ support; and/or for treating acidosis in particular without (increased) administration of bicarbonate. Such compositions are particularly useful for regenerating carrier protein-containing multiple pass dialysis fluids having pH values from 6.35 to 11.4, in particular from 6.5 to 10, preferably from 7.4 to 9, when entering the dialyzer. It is also an object of the present invention to provide a method for regenerating a carrier protein-containing multiple pass dialysis fluid, which can be used for a variety of dialysis procedures, in particular for dialysis procedures requiring pH values from 6.35 to 11.4, in particular from 6.5 to 10, preferably from 7.4 to 9.

This object is achieved by means of the subject-matter set out below and in the appended claims.

Although the present invention is described in detail below, it is to be understood that this invention is not limited to the particular methodologies, protocols and reagents described herein as these may vary. It is also to be understood that the terminology used herein is not intended to limit the scope of the present invention which will be limited only by the appended claims. Unless defined otherwise, all technical and scientific terms used herein have the same meanings as commonly understood by one of ordinary skill in the art.

In the following, the elements of the present invention will be described. These elements are listed with specific embodiments, however, it should be understood that they may be combined in any manner and in any number to create additional embodiments. The variously described examples and preferred embodiments should not be construed to limit the present invention to only the explicitly described embodiments. This description should be understood to support and encompass embodiments which combine the explicitly described embodiments with any number of the disclosed and/or preferred elements. Furthermore, any permutations and combinations of all described elements in this application should be considered disclosed by the description of the present application unless the context indicates otherwise.

Throughout this specification and the claims which follow, unless the context requires otherwise, the terms “comprise” and “contain”, and variations such as “comprises” and “comprising” or “contains” and “containing”, will be understood to imply the inclusion of a stated member, integer or step but not the exclusion of any other non-stated member, integer or step. The term “consist of” is a particular embodiment of the term “comprise” or “contain”, wherein any other non-stated member, integer or step is excluded. In the context of the present invention, the terms “comprise” and “contain” encompass the term “consist of”. The terms “comprising” and “containing” thus encompasses “consisting of” e.g., a composition “comprising”/“containing” X may consist exclusively of X or may include something additional e.g., X+Y.

The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. No language in the specification should be construed as indicating any non-claimed element essential to the practice of the invention.

The word “substantially” does not exclude “completely” e.g., a composition which is “substantially free” from Y may be completely free from Y. Where necessary, the word “substantially” may be omitted from the definition of the invention.

The term “about” in relation to a numerical value x means x±10%.

Kit for Treating a Carrier-Protein Containing Multiple Pass Dialysis Fluid

In a first aspect the present invention provides a kit for treating a carrier protein-containing multiple pass dialysis fluid comprising
(a) an acidic composition comprising a biologically compatible acid, and
(b) an alkaline composition comprising a biologically compatible base, wherein the ratio of the concentration of the biologically compatible acid in the acidic composition (a) to the concentration of the biologically compatible base in the alkaline composition (b) is in the range from 0.7 to 1.3, preferably in the range from 0.75 to 1.25 and more preferably in the range from 0.8 to 1.2 and wherein the concentration of the biologically compatible acid in the acidic composition and the concentration of the biologically compatible base in the alkaline composition is at least 50 mmol/l and no more than 500 mmol/l.

Such a kit (i) enables the “regeneration” (in particular the “cleaning”) of a carrier protein, in particular albumin, carrying a toxin by modification of the pH value, (ii) provides the required electrolytes and/or nutrients and (iii) enables an adjustment of the pH of the dialysis fluid to values from 6.35 to 11.4, in particular from 6.5 to 10, preferably from 7.4 to 9. Thus, such compositions can be used in different dialysis procedures, e.g. for renal support, liver support, lung support; for multiple organ support; for the regulation of the acid-base homeostasis; and/or for treating acidosis, in particular for regenerating carrier protein-containing, in particular albumin containing, multiple pass dialysis fluids having pH values from 6.35 to 11.4, in particular from 6.5 to 10, preferably from 7.4 to 9.

The term “regenerating” as used herein (i.e. throughout the specification), in particular in the context of “regenerating a carrier protein, such as albumin”, means that after passing the dialyzer substances, which are to be removed from the blood, such as toxins, are bound to the carrier protein. These substances need to be released from the carrier protein in order to reuse the carrier protein in the next cycle of a multiple-pass dialysis. Accordingly, “regenerating” (a carrier protein) means that the carrier protein is transferred from a state (X), in which toxins or other substances to be removed are bound to the carrier protein, to a state (Y), in which the carrier protein is “unbound” (or free). In particular, in such an unbound state (Y) the carrier protein has a conformation enabling the carrier protein to bind to toxins and other substances to be removed from the blood.

The term “treating a carrier protein-containing multiple pass dialysis fluid”, as used herein (i.e. throughout the specification), in general refers to (i) bringing each of the constituents of the kit according to the present invention (e.g. each of the acidic composition (a), the alkaline composition (b), and any further optional constituent such as compositions (c1)-(c12) as described herein) in contact with a carrier protein-containing multiple pass dialysis fluid, thereby (ii) influencing the properties of the carrier protein-containing multiple pass dialysis fluid, for example changing the pH value of the dialysis fluid, changing the composition of the dialysis fluid, and/or—most preferably—regenerating the carrier protein. In this context, the term “treating a carrier protein-containing multiple pass dialysis fluid”, as used herein (i.e. throughout the specification), refers preferably to regenerating the carrier protein in the carrier protein-containing multiple pass dialysis fluid as described above. In particular, each of the constituents of the kit according to the present invention (e.g. each of the acidic composition (a), the alkaline composition (b), and any further optional constituent such as compositions (c1)-(c12) as described herein) is directly added to the carrier protein-containing multiple pass dialysis fluid. Preferably, each of the constituents of the kit according to the present invention (e.g. each of the acidic composition (a), the alkaline composition (b), and any further optional constituent such as compositions (c1)-(c12) as described herein) is added to the carrier protein-containing multiple pass dialysis fluid directly in a separate manner. In other words, the constituents of the kit (e.g. the acidic composition (a), the alkaline composition (b), and any further optional constituent such as compositions (c1)-(c12) as described herein) are preferably not mixed with each other before they are brought in contact with (e.g. added to) the carrier-protein-containing multiple pass dialysis fluid.

The term “carrier-protein-containing multiple pass dialysis fluid”, as used herein, refers to a dialysis fluid, which (i) repeatedly passes the dialyzer (and is thus repeatedly used for dialyzing blood), preferably in a continuous manner, and (ii) comprises a carrier-protein, i.e. a protein, which is involved in the movement of ions, small molecules or macromolecules. In particular, the carrier protein in the dialysis fluid enables the removal of toxic and/or undesirable ions, small molecules or macromolecules from the blood during dialysis. The carrier protein is preferably a water-soluble protein. In the context of the present invention as described herein a preferred carrier protein is albumin, preferably serum albumin, more preferably mammalian serum albumin, such as bovine or human serum albumin and even more preferably human serum albumin (HSA). Albumin may be used as it occurs in nature or may be genetically engineered albumin. Mixtures containing albumin and at least one further carrier protein and mixtures of different types of albumin, such as a mixture of human serum albumin and another mammalian serum albumin, are also preferred. In any case, the albumin concentration specified herein refers to the total concentration of albumin, no matter if one single type of albumin (e.g. human serum albumin) or a mixture of various types of albumin is used. The dialysis fluid used in the present invention comprises 3 to 80 g/l albumin, preferably 12 to 60 g/l albumin, more preferably 15 to 50 g/l albumin, and most preferably about 20 g/l albumin. The concentration of albumin can also be indicated as % value and, thus, for example 30 g/l albumin correspond to 3% albumin (wt./vol).

Preferably, the kit according to the present invention as described herein does not comprise a carrier-protein such as albumin.

Preferably, in the kit according to the present invention as described herein the acidic composition (a) and the alkaline composition (b) are provided in a spatially separated manner, for example in separate containers. More preferably, the kit according to the present invention as described herein comprises a first container comprising the acidic composition (a) (but not the alkaline composition (b)) and a second container comprising the alkaline composition (b) (but not the acidic composition (a)).

The kit according to the present invention comprises (a) an acidic composition comprising a biologically compatible acid. Preferably, the acidic composition (a) comprises or consists of an aqueous solution of a biologically compatible acid. The term “acid” as used herein refers to Arrhenius acids, i.e., acids that dissociate in solution to release hydrogen ions (H⁺). A “biologically compatible acid”, as used herein, refers to any acid, which—if comprised by a dialysis fluid, which also comprises a biologically compatible base as described herein—does not exert toxic or injurious effects to the subject treated with dialysis, in particular does not exert toxic or injurious effects to the dialyzed blood. Non-limiting examples of a biologically compatible acid include (i) strong inorganic acids such as hydrochloric, sulfuric, sulfamic and nitric acid; (ii) organic acids such as acetic acid, benzoic acid, oxalic acid, citric acid, hippuric acid, glucuronic acid, uric acid, glutamic acid, aspartic acid, m-hydroxyhippuric acid, p-hydroxyphenyl-hydracrylic acid, aminoisobutyric acid, formic acid, pyruvic acid, ascorbic acid, oxoglutaric acid, guanidinoacetic acid, dehydroascorbic acid, aminoisobutyric acid, fumaric acid, glycolic acid, lactic acid, malic acid, maleic acid and tartaric acid, as well as (iii) acids such as sodium and potassium bisulfate (NaHSO₄ and KHSO₄), potassium acid phthalate, indoxyl sulfuric acid and phosphoric acid. Moreover, the term “biologically compatible acid” also refers to mixtures of acids, such as mixtures of the above exemplified acids. Preferably, the acidic composition (a) comprises hydrochloric, sulfuric and/or acetic acid. Accordingly, the acidic composition (a) preferably comprises or consists of an aqueous solution of hydrochloric, sulfuric and/or acetic acid. More preferably, the acidic composition (a) comprises or consists of an aqueous solution of hydrochloric acid. Hydrochloric acid has the advantage that the result from a combination with sodium hydroxide (e.g. as biologically compatible base in the alkaline composition (b) of the kit of the present invention) is sodium chloride.

In the acidic composition (a), the biologically compatible acid may be dissociated and/or undissociated, e.g. completely dissociated, partly dissociated/undissociated or completely undissociated. Typically, in the acidic composition (a), the biologically compatible acid is partly or completely dissociated. The acidic composition (a) may be in solid, e.g. powder, gel, partially crystalline, gas phase or liquid physical condition. Preferably, the acidic composition is a liquid, such as an aqueous solution of the biologically compatible acid.

Reactions of acids are often generalized in the form HA⇄H⁺+A⁻, with HA representing the acid and A⁻ the conjugate base. It is also possible that the acid can be the charged species and the conjugate base can be neutral (reaction scheme: HA⁺⇄H⁺+A. In solution there is typically an equilibrium between the acid and its conjugate base. The acid dissociation constant Kₐ is generally used in the context of acid-base reactions. The numerical value of K, is equal to the product of the concentrations of the products divided by the concentration of the reactants, where the reactant is the acid (HA) and the products are the conjugate base and H⁺. In other words,

$$Ka = \frac{[ {A}^{-} ] [ {H}^{+} ]}{[ A H ]} ,$$

wherein the brackets indicate the concentration (i.e. [A⁻] means concentration of the conjugate base, etc.).

The stronger the acid, the higher the Kₐ, since the ratio of hydrogen ions to acid is typically higher for the stronger acid (as the stronger acid has a greater tendency to lose its proton). Because the range of possible values for Kₐ spans many orders of magnitude, a more manageable constant, pKₐ is more frequently used, where pKₐ=−log₁₀Kₐ. Stronger acids have a smaller pKₐ than weaker acids. Typically pKₐ values given are those pKₐ values, which are experimentally determined pKₐ at 25° C. in aqueous solution. The pKₐ value of the biologically compatible acid comprised by the acidic composition (a) is preferably in the range from −6.5 to 6.5, more preferably in the range from −6.5 to 5.0.

Preferably, the acidic composition (a) has a pH in the range from 0.5 to 3.0, preferably in the range from 0.7 to 2.0, more preferably in the range from 0.9 to 1.2 and most preferably in the range from 1.0 to 1.1, for example about 1.05. The carrier protein comprised by the carrier protein-containing multiple pass dialysis fluid unfolds in extremely acidic pH values, thereby releasing the carried substance, e.g. a toxin. The free-floating toxin can then be easily removed, e.g. by filtration. On the other hand, exposure of the carrier protein to an extremely acidic pH value may result in denaturation of the carrier protein. Intensive testing has revealed that a pH value of the dialysis fluid, which is in the range from 1.5 to 5, preferably in the range from 1.8 to 4.5 and more preferably in the range from 2.3 to 4, enables sufficient removal of the toxins and avoids denaturation of the carrier protein. Such a pH value of the dialysis fluid is obtained by addition of an acidic composition (a) having a pH in the range from 0.5 to 3.0, preferably in the range from 0.7 to 2.0, more preferably in the range from 0.9 to 1.2 and most preferably in the range from 1.0 to 1.1, for example about 1.05, to the dialysis fluid (which has a pH in the range from 6.35 to 11.4, in particular from 6.5 to 10, preferably from 7.4 to 9, before adding the acidic composition (a)).

In particular to obtain such a pH value as described above, the concentration of the biologically compatible acid, in particular the concentration of HCl, in the acidic composition (a) may be adjusted accordingly. For example, the biologically compatible acid may be provided diluted or undiluted. Preferably, the biologically compatible acid is diluted in the acidic composition (a). Accordingly, it is more preferred that the acidic composition (a) is a solution of the biologically compatible acid (optionally comprising further components). Even more preferably, the acidic composition (a) is an aqueous solution of the biologically compatible acid (optionally comprising further components).

The concentration of the biologically compatible acid, in particular the concentration of HCI, in the acidic composition (a) is at least 50 mmol/l, preferably at least 60 mmol/l, more preferably at least 70 mmol/l, even more preferably at least 80 mmol/l and most preferably at least 100 mmol/l.

The concentration of the biologically compatible acid, in particular the concentration of HCl, in the acidic composition (a) may be, for example, 6200 mmol/l. Preferably, the concentration of the biologically compatible acid, in particular the concentration of HCI, in the acidic composition (a) is no more than 0.5 mol/l, more preferably no more than 0.4 mol/l, even more preferably no more than 0.3 mol/l and most preferably no more than 0.2 mol/l.

Accordingly, the concentration of the biologically compatible acid, in particular of HCl, in the acidic composition (a) is preferably from 50 mmol/l to 6.20 mol/l, more preferably from 60 mmol/l to 0.4 mol/l, even more preferably from 70 mmol/l to 0.3 mol/l and most preferably from 100 mmol/l to 200 mmol/l.

The acidic composition (a) is preferably added directly (i.e. without further modifications, in particular undiluted) to a carrier protein-containing multiple pass dialysis fluid as described herein. More preferably, the acidic composition (a) comprising the biologically compatible acid is an aqueous solution of the biologically compatible acid (optionally comprising further components), which can be directly added to the dialysis fluid as described herein.

Preferably, the acidic composition (a) comprises further components, in addition to the biologically compatible acid and, optionally, H₂O. Thereby, a stabilizer for a carrier protein, in particular albumin, an electrolyte and/or a nutrient as described below are preferred further components. More preferably, the acidic composition (a) further comprises electrolytes as described herein. It is also preferred that the acidic composition (a) does not comprise a stabilizer for a carrier protein, a nutrient and/or bicarbonate. Alternatively, it is also preferred that the acidic composition (a) does not comprise any further components in addition to the biologically compatible acid and optionally H₂O.

The kit according to the present invention further comprises (b) an alkaline composition comprising a biologically compatible base. Preferably, the alkaline composition (b) comprises or consists of an aqueous solution of a biologically compatible base. The term “base” as used herein refers to Arrhenius bases, i.e., bases that dissociate in solution to release hydroxide ions (OH⁻). A “biologically compatible base”, as used herein, refers to any base, which—if comprised by a dialysis fluid, which also comprises a biologically compatible acid as described herein—does not exert toxic or injurious effects to the subject treated with dialysis, in particular does not exert toxic or injurious effects to the dialyzed blood. Non-limitingexamples of a biologically compatible base include sodium hydroxide, potassium hydroxide, magnesium hydroxide and calcium hydroxide. Moreover, the term “biologically compatible base” also refers to mixtures of bases, such as mixtures of the above exemplified bases. Preferably, the alkaline composition (b) comprises sodium hydroxide and/or potassium hydroxide. Thereby, the alkaline composition (b) preferably comprises or consists of an aqueous solution of sodium hydroxide and/or potassium hydroxide. More preferably, the alkaline composition (b) comprises or consists of an aqueous solution of sodium hydroxide. Sodium hydroxide has the advantage that the result from a combination with hydrochloric acid (e.g. as biologically compatible acid in the acidic composition (a) of the kit of the present invention) is sodium chloride.

In the alkaline composition (b), the biologically compatible base may be dissociated and/or undissociated, e.g. completely dissociated, partly dissociated/undissociated or completely undissociated. Typically, in the alkaline composition (b), the biologically compatible base is partly or completely dissociated. The alkaline composition (b) may be in solid, e.g. powder, gel, partially crystalline, gas phase or liquid physical condition. Preferably, the alkaline composition is a liquid, such as an aqueous solution of the biologically compatible base.

Reactions of bases are often generalized in the form B+H₂O⇄O═OH⁻+BH⁺, with B representing the base and BH⁺ its acid. The base dissociation constant K_{b} is generally used in the context of acid-base reactions. The numerical value of K_{b} is equal to the product of the concentrations of the products divided by the concentration of the reactants, where the reactant is the base (B) and the products are its conjugate acid (BH⁺) and OH⁻. In other words,

$${K}_{b} = \frac{[ {BH}^{+} ] * [ {OH}^{-} ]}{[ B ]}$$

wherein the brackets indicate the concentration (i.e. [OH⁻] means concentration of the hydroxide ions, etc.).

The stronger the base, the higher the K_{b}. Because the range of possible values for K_{b} spans many orders of magnitude, a more manageable constant, pK_{b} is more frequently used, where pK_{b}=−log₁₀ K_{b}. Stronger bases have a smaller pK_{b} than weaker bases. Typically, pK_{b} values given are those pK_{b} values, which are experimentally determined at 25° C. in aqueous solution. The pK_{b} value of the biologically compatible base comprised by the alkaline composition (b) is preferably in the range from −6.5 to 6.5, more preferably in the range from −6.5 to 5.0.

Preferably, the alkaline composition (b) has a pH in the range from 10.0 to 14.0, preferably in the range from 11.5 to 13.5, more preferably in the range from 12.0 to 13.0 and most preferably in the range from 12.3 to 12.9, for example about 12.6. The carrier protein comprised by the carrier protein-containing multiple pass dialysis fluid unfolds in extremely alkaline pH values, thereby releasing the carried substance, e.g. a toxin. The free-floating toxin can then be easily removed, e.g. by filtration. On the other hand, exposure of the carrier protein to an extremely alkaline pH value may result in denaturation of the carrier protein. Intensive testing has revealed that a pH value of the dialysis fluid, which is in the range from 9.5 to 12.5, preferably in the range from 10.5 to 12.0 and more preferably in the range from 11 to 11.5, enables sufficient removal of the toxins and avoids denaturation of the carrier protein. Such a pH value of the dialysis fluid is obtained by addition of an alkaline composition (b) having a pH in the range from 10.0 to 14.0, preferably in the range from 11.5 to 13.5, more preferably in the range from 12.0 to 13.0 and most preferably in the range from 12.3 to 12.9, for example about 12.6, to the dialysis fluid (which has a pH in the range from 6.35 to 11.4, in particular from 6.5 to 10, preferably from 7.4 to 9, before adding the alkaline composition (b)).

In particular to obtain such a pH value as described above, the concentration of the biologically compatible base, in particular the concentration of NaOH, in the alkaline composition (b) may be adjusted accordingly. For example, the biologically compatible base may be provided diluted or undiluted. Preferably, the biologically compatible base is diluted in the alkaline composition (b). Accordingly, it is more preferred that the alkaline composition (b) is a solution of the biologically compatible base (optionally comprising further components). Even more preferably, the alkaline composition (b) is an aqueous solution of the biologically compatible base (optionally comprising further components).

The concentration of the biologically compatible base, in particular the concentration of NaOH, in the alkaline composition (b) is at least 50 mmol/l, preferably at least 60 mmol/l, more preferably at least 70 mmol/l, even more preferably at least 80 mmol/l and most preferably at least 100 mmol/l.

The concentration of the biologically compatible base, in particular the concentration of NaOH, in the alkaline composition (b) may be, for example, 6200 mmol/l. Preferably, the concentration of the biologically compatible base, in particular the concentration of NaOH, in the alkaline composition (b) is no more than 0.5 mol/l, more preferably no more than 0.4 mol/l, even more preferably no more than 0.3 mol/l and most more preferably no more than 0.2 mol/l.

Accordingly, the concentration of the biologically compatible base, in particular of NaOH, in the alkaline composition (b) is preferably from 50 mmol/l to 6.20 mol/l, more preferably from 60 mmol/l to 0.4 mol/l, even more preferably from 70 mmol/l to 0.3 mol/l and most preferably from 100 mmol/l to 200 mmol/l.

The alkaline composition (b) is preferably added directly (i.e. without further modifications, in particular undiluted) to a carrier protein-containing multiple pass dialysis fluid as described herein. More preferably, the alkaline composition (b) comprising the biologically compatible base is an aqueous solution of the biologically compatible base (optionally comprising further components), which can be directly added to the dialysis fluid as described herein.

Preferably, the alkaline composition (b) comprises further components, in addition to the biologically compatible base and, optionally, H₂O. Thereby, a stabilizer for a carrier protein, in particular albumin, an electrolyte and/or a nutrient as described below are preferred further components. More preferably, the alkaline composition (b) further comprises a stabilizer for a carrier protein as described herein and/or electrolytes as described herein, but preferably not magnesium and/or calcium. It is also preferred that the alkaline composition (b) does not comprise a nutrient and/or magnesium and/or calcium. Alternatively, it is also preferred that the alkaline composition (b) does not comprise any further components in addition to the biologically compatible base and optionally H₂O.

In the kit according to the present invention, the ratio of the concentration of the biologically compatible acid in the acidic composition (a) to the concentration of the biologically compatible base in the alkaline composition (b) is in the range from 0.7 to 1.3, preferably in the range from 0.75 to 1.25 and more preferably in the range from 0.8 to 1.2. Such a ratio ensures that the pH value of the dialysis fluid passing the dialyzer can be adjusted to values from 6.35 to 11.4, in particular to values from 6.5 to 10, preferably to values from 7.4 to 9.

Preferably, the kit comprises a stabilizer for a carrier protein, in particular a stabilizer for albumin. A stabilizer for a carrier protein, in particular a stabilizer for albumin, prolongs the lifetime of the carrier protein, in particular of albumin. In each dialysis cycle, the carrier protein, in particular albumin, undergoes a treatment with the acidic composition (a) and/or with the alkaline composition (b) in order to regenerate the carrier protein. Regeneration of the carrier protein (such as albumin) is achieved by exposing the carrier protein (such as albumin) to extremely acidic and alkaline pH values as provided by the acidic composition (a) and the alkaline composition (b) as described herein, thereby unfolding the carrier protein (such as albumin) and thus releasing the carried substance, e.g. a toxin. However, repeated folding/unfolding of the carrier protein, in particular of the albumin molecule, (e.g. in each dialysis/regeneration cycle) may result in irreversible denaturation of the carrier protein, in particular of the albumin molecule. A stabilizer for a carrier protein, in particular a stabilizer for albumin, protects the carrier protein, in particular albumin, from such irreversible denaturation and a single carrier protein molecule can undergo more dialysis/regeneration cycles with a stabilizer than in absence of a stabilizer. Therefore, when the carrier protein is regenerated by treatment with an acidic composition (a) and an alkaline composition (b), the stabilizer protects the carrier protein from irreversible denaturation and, thus, prolongs the lifetime of the carrier protein.

The stabilizer for a carrier protein, in particular the stabilizer for albumin, is preferably comprised by the alkaline composition (b) or by a (separate) stabilizer composition (c1). “(Separate) stabilizer composition (c1)” means that this composition is distinct from the acidic composition (a) and from the alkaline composition (b).

Preferably, the stabilizer for a carrier protein, in particular the stabilizer for albumin, is not comprised by the acidic composition (a).

Particularly preferably, the kit according to the present invention as described herein comprises
(c1) a stabilizer composition comprising the stabilizer for a carrier protein, in particular the stabilizer for albumin,
   wherein the stabilizer composition (c1) is different from the acidic composition (a) and from the alkaline composition (b).

It is thus preferred that the stabilizer composition (c1) is provided in a spatially separated manner, for example in a container, which comprises the stabilizer composition (c1) (but which container neither comprises the acidic composition (a) nor the alkaline composition (b)).

The stabilizer composition (c1) may be in solid, e.g. powder, in gel, in partially crystalline, in gas phase or in liquid physical condition. Preferably, the stabilizer composition is a liquid, such as a solution, in particular an aqueous solution, comprising the stabilizer for a carrier protein, in particular the stabilizer for albumin.

The stabilizer for a carrier protein, in particular the stabilizer for albumin, is typically a protein stabilizer. Protein stabilizers are known in the art and, as such, commercially available. In general, protein stabilizers increase the stability of proteins in solutions. As used herein, the term “protein stabilizer” refers to any compound having the ability to change a protein's reaction equilibrium state, such that the native state of the protein is improved or favored. Moreover, when selecting a protein stabilizer, the skilled person is aware that the binding of the toxins to be removed to the carrier protein, in particular to albumin, must not be strengthened by the protein stabilizer in such a way that very extreme pH values, which would destroy the carrier protein, in particular albumin, were required to release the toxin from the carrier protein, in particular albumin.

Examples of protein stabilizers useful in the context of the present invention include, but are not limited to, sugars such as sucrose, sorbitol or glucose; polyhydric alcohols such as glycerol or sorbitol; polymers such as polyethylene glycol (PEG) and a-cyclodextrin; amino acids such as arginine, proline, and glycine and/or salts thereof; fatty acids and/or salts thereof; osmolytes; and Hoffmeister salts such as Tris, sodium sulfate and potassium sulfate; and derivatives and structural analogs thereof. Moreover, also combinations thereof may serve as protein stabilizers. In general, a protein stabilizer selected from the group consisting of fatty acids, amino acids, sugars and osmolytes is preferred; a protein stabilizer selected from the group consisting of fatty acids, amino acids and sugars is more preferred; a protein stabilizer selected from the group consisting of fatty acids and amino acids is even more preferred; and a protein stabilizer, which is a fatty acid is most preferred.

As used herein, the term “derivative” refers to a compound, which is derived from a reference compound by a (single) chemical reaction. Typically, a “derivative” can (at least theoretically) be formed from a (precursor) compound (with the precursor compound being the reference compound). A derivative is different from a “structural analog”, which refers to a compound that can be imagined to arise from a reference compound, if an atom or a group of atoms, such as a functional group, is replaced with another atom or group of atoms, such as a functional group. For example, in a “structural analog” a functional group may be replaced by another functional group, preferably without changing the “function” of the functional group. The term “functional group” as used herein refers to specific groups (moieties) of atoms or bonds within molecules that are responsible for the characteristic chemical reactions of those molecules. Typically, the same functional group will undergo the same or similar chemical reaction(s) regardless of the size of the molecule it is a part of, although its relative reactivity can be modified by other functional groups nearby.

In the kit according to the present invention, the stabilizer for a carrier protein, in particular the stabilizer for albumin, is preferably selected from the group consisting of amino acids, salts of amino acids, derivatives of amino acids, fatty acids, salts of fatty acids, derivatives of fatty acids, sugars, polyols and osmolytes.

Among amino acids, small neutral amino acids, such as alanine, serine, threonine, proline, methionine, valine and glycine, are preferred. Such small neutral amino acids exhibit a concentration-independent degree of preferential hydration and therefore belong to preferred protein stabilizers. Moreover, a preferred stabilizer in the kit according to the present invention is acetyl tryptophan or tryptophan. A modified amino acid, e.g. having increased shelf life, is also preferred, such as acetyl tryptophan.

Sugars also increase the hydration status thereby preventing denaturation. Among sugars, sucrose, sorbitol, glucose, dextran and mannitol are preferred. Sorbitol and dextran are more preferred.

Among osmolytes a preferred protein stabilizer may be selected from the group consisting of taurine, betaine, glycine and sarcosine. More preferably, a protein stabilizer may be selected among osmolytes from the group consisting of taurine, glycine and sarcosine. Even more preferably, a protein stabilizer may be selected among osmolytes from taurine and sarcosine. The most preferable osmolyte as a protein stabilizer is taurine.

A particularly preferred stabilizer in the kit according to the present invention is selected from the group consisting of fatty acids, salts of fatty acids and derivatives of fatty acids. Preferred fatty acids (and salts or derivatives thereof) are saturated or unsaturated fatty acids (and salts or derivatives thereof) having no more than 20 carbon atoms, such as caprylic acid, capric acid, lauric acid, oleic acid and palmitic acid (and salts or derivatives thereof); more preferably are saturated or unsaturated fatty acids (and salts or derivatives thereof) having no more than 15 carbon atoms such as caprylic acid, capric acid and lauric acid (and salts or derivatives thereof); and even more preferred are saturated or unsaturated fatty acids (and salts or derivatives thereof) having no more than 13 carbon atoms, such as caprylic acid, capric acid and lauric acid (and salts or derivatives thereof).

Fatty acids can exert antimicrobial effects and, thus, prevent the growth of pathogenic microbes in the dialysis device. Preferably, the stabilizer is selected from the group consisting of caprylic acid, capric acid, lauric acid, oleic acid and palmitic acid and salts or derivatives thereof. More preferably, the stabilizer is selected from the group consisting of caprylic acid, capric acid, lauric acid and oleic acid and salts or derivatives thereof. Even more preferably the stabilizer is selected from the group consisting of caprylic acid, capric acid and lauric acid and salts or derivatives thereof. Most preferably, the stabilizer is selected from the group consisting of caprylic acid and capric acid and salts or derivatives thereof; in particular from the group consisting of caprylate, caprylic acid, caprate, capric acid, caproic acid and caproate. Particularly preferably the stabilizer is a caprylate, for example sodium caprylate (C₈H₁₅NaO₂). In other words, in view of prevention of denaturation, biocompatibility, solubility and improvement of detoxification, caprylate is the most preferred protein stabilizer. In addition, caprylate prevents bacterial growth at least during 24 hours treatment in a recirculating dialysis fluid.

Preferably, the concentration of the stabilizer for a carrier protein, in particular when comprised by a composition, which is different from the acidic composition (a) and different from the alkaline composition (b), such as the stabilizer composition (c1) or the stabilizer/nutrient composition (c5), is in the range from 1 to 2500 mmol/l, preferably from 37 to 2020 mmol/l, more preferably from 50 to 1500 mmol/l, even more preferably from 100 to 1000 mmol/l and most preferably from 150 to 500 mmol/l.

If the stabilizer for a carrier protein, in particular the stabilizer for albumin, is comprised by the alkaline composition (b), the concentration of the stabilizer in the alkaline composition (b) is preferably in the range from 0.01 mmol/l to 200 mmol/l, more preferably from 0.1 to 100 mmol/l, even more preferably from 0.5 to 50 mmol/l and most preferably from 1 to 10 mmol/l.

It is also preferred that the kit according to the present invention comprises a nutrient. The term “nutrient”, as used herein, refers to a substance used in an organism's metabolism. Preferred examples of nutrients include proteins or amino acids, trace elements, vitamins such as lipo-soluble or water-soluble vitamins, carbohydrates such as sugars and combinations thereof. Preferred nutrient amino acids are, for example, the essential amino acids phenylalanine, valine, threonine, tryptophan, methionine, leucine, isoleucine, lysine and histidine. A “trace element”, as used herein, refers to a dietary element that is needed in very minute quantities for the proper growth, development and physiology of an organism. Examples of trace elements include boron, cobalt, chromium, copper, fluoride, iodine, iron, manganese, molybdenum, selenium and zinc. Examples of vitamins include vitamin A (retinol), vitamin B₁ (thiamin), vitamin B₂ (riboflavin), vitamin B₃ (niacin), vitamin B₅ (pantothenic acid), vitamin B₆ (pyridoxine, pyridoxal, and pyridoxamine), vitamin B₇ (biotine), vitamin B₈ (ergadenylic acid), vitamin B₉ (folic acid), vitamin B₁₂ (cyanocobalamin), vitamin C (ascorbic acid), vitamin D, vitamin E (tocopherol), vitamin K, choline and carotenoids such as alpha carotene, beta carotene, cryptoxanthin, lutein, lycopene and zeaxanthin.

More preferably, the kit according to the present invention comprises a sugar. The term “sugar”, as used herein, refers to short-chain carbohydrates, which are typically soluble. The term “sugar” includes monosaccharides such as glucose, fructose and galactose;

disaccharides such as sucrose, maltose, trehalose and lactose; and oligosaccharides, which are saccharide polymers having a small number—typically three to nine—of monosaccharides. The kit according to the present invention may comprise one or more of the above sugars, i.e. alone or combinations thereof.

Moreover, if the kit according to the present invention comprises one or more sugars, it preferably further comprises one or more proteins or amino acids as described above. Moreover, if the kit according to the present invention comprises one or more sugars, it preferably further comprises one or more trace elements as described above. Moreover, if the kit according to the present invention comprises one or more sugars, it preferably further comprises one or more vitamins as described above.

Preferably, the sugar comprised by the kit according to the present invention is glucose. More preferably, glucose is the only sugar comprised by the kit according to the present invention. Even more preferably, glucose is the only nutrient comprised by the kit according to the present invention. Most preferably, the glucose is D-glucose.

Preferably, the nutrient as described herein, in particular the sugar, is neither comprised by the acidic composition (a) nor by the alkaline composition (b). Therefore, it is preferred that the kit according to the present invention comprises
(c2) a nutrient composition comprising a nutrient, in particular a sugar,
   wherein the nutrient composition (c2) is different from the acidic composition (a) and from the alkaline composition (b).

It is thus preferred that the nutrient composition (c2) is provided in a spatially separated manner, for example in a container, which comprises the nutrient composition (c2) (but which container neither comprises the acidic composition (a) nor the alkaline composition (b)).

Thus, a kit according to the present invention preferably comprises a stabilizer composition (c1) and a nutrient composition (c2), wherein the stabilizer composition (c1) and the nutrient composition (c2) may be the same composition (c5) or distinct compositions. Preferably, the nutrient composition (c2) is the same composition as the stabilizer composition (c1). In other words, it is preferred that the kit according to the present invention comprises a nutrient/stabilizer composition (c5), which comprises both, the stabilizer as described above and the nutrient, in particular the sugar, as described above. Preferably, the nutrient/stabilizer composition (c5) is provided in a spatially separated manner, for example in a container, which comprises the nutrient/stabilizer composition (c5) (but which container neither comprises the acidic composition (a) nor the alkaline composition (b)).

Preferably, the sugar comprised by the nutrient composition (c2) (or the stabilizer/nutrient composition (c5)) is glucose as described above.

The nutrient composition (c2) (or the stabilizer/nutrient composition (c5)) may be in solid, e.g. powder, in gel, in partially crystalline, in gas phase or in liquid physical condition. Preferably, the nutrient composition is a liquid, such as a solution, in particular an aqueous solution, comprising the nutrient, in particular glucose.

The concentration of the nutrient, preferably sugar, in particular glucose, in particular when comprised by a composition, which is different from the acidic composition (a) and different from the alkaline composition (b), such as the nutrient composition (c2) or the stabilizer/nutrient composition (c5), is preferably in the range from 100 to 3500 mmol/l, more preferably in the range from 160 to 2780 mmol/l, even more preferably in the range from 200 to 2500 mmol/l and most preferably in the range from 250 to 2280 mmol/l.

It is particularly preferred that the kit according to the present invention comprises a nutrient/stabilizer composition (c5), which comprises a nutrient, preferably glucose, and a stabilizer for a carrier protein, preferably a caprylate, and wherein the composition (c5) is different from the acidic composition (a) and from the alkaline composition (b).

Preferably, the kit according to the present invention comprises at least one component selected from the group consisting of sodium, chloride, calcium, magnesium, potassium, phosphate and carbonate/bicarbonate (hydrogen carbonate). Preferably, such a component is provided as ions, i.e. the kit according to the present invention preferably comprises sodium ions (Na⁺), chloride ions (Cl⁻), calcium ions (Ca²⁺), magnesium ions (Mg²⁺), potassium (K⁺), phosphate ions (H₂PO₄⁻, HPO₄²⁻ or PO₄³⁻) and/or (hydrogen) carbonate ions (CO₃²⁻, HCO₃⁻).

Human blood contains many components. Dialysis patients often suffer from a deficit or excess of electrolytes, which is to be compensated by dialysis. This is achieved on the one hand by a concentration gradient between blood and dialysis fluid and on the other hand by filtration. The dialysis fluid thus preferably comprises (i) electrolytes, (ii) a bicarbonate buffer system and/or (iii) glucose as described above. Therefore, components such as sodium, potassium, calcium, magnesium, chloride ions, glucose and a buffer are preferably included in the kit according to the present invention.

Preferably, the kit according to the present invention does not comprise calcium, magnesium, and carbonate/bicarbonate (hydrogen carbonate), in particular the kit according to the present invention does preferably not comprise calcium ions (Ca²⁺), magnesium ions (Mg²⁺), and (hydrogen) carbonate ions (CO₃²⁻, HCO₃⁻). In the absence of those three components, the kit can be used to obtain/regenerate a carrier protein-containing multiple pass dialysis fluid having a pH in the range of 6.35 to 11.4, in particular in the range of 6.5 to 10, preferably in the range of 7.4 to 9, i.e. for a dialysis fluid having an even wider range of pH-values.

Preferably, the kit according to the present invention comprises sodium, in particular sodium ions. The minimum sodium concentration in a patient's blood is typically 133-135 mmol/l in the physiological range (pathological minimum: 120 mmol/l). Increases or decreases in sodium concentration have to be performed very slowly, as dialyzing a patient against the wrong sodium concentration can be very harmful for a patient: hypotension or brain oedema can be the consequences. In order to enable a dialysis of patients having a very low sodium concentration in the blood, often a dialysis fluid is chosen, which has a sodium concentration as low as possible. To adapt the dialysis fluid for patients with higher sodium levels, additional sodium can be provided.

Preferably, the source of sodium, in particular of sodium ions, is NaOH, Na₂CO₃, Na₂HPO₄, NaHCO₃, NaCl, and/or a sodium salt of lactate, acetate, gluconate, citrate, maleate, tartrate and/or of fatty acids such as caprylate. Preferably, in the kit according to the present invention the major source of sodium is NaOH.

For example, a component, such as sodium, may be provided in the acidic composition (a), in the alkaline composition (b) or in any other composition/constituent of the kit. Such compositions may be in solid or liquid physical condition. If the component, such as sodium, is comprised by a liquid composition, it is typically an ion derived from a certain substance, for example a sodium ion derived from (dissociated) NaCl, NaOH etc. (as described above). Accordingly, the “source of . . . ”, as used herein, refers to the substance from which an ion is derived.

Preferably, the kit according to the present invention comprises chloride, in particular chloride ions. Preferably, the source of chloride, in particular of chloride ions, is HCl, NaCl, KCl, MgCl₂, and/or CaCl₂.

The chloride concentration of the patients should be kept in the physiological range. A preferred source of chloride is HCl. If the chloride concentration is to be kept low, sodium salts other than NaCl can be used as source of sodium, as described above. However, high concentrations of buffers (e.g. Na₂CO₃, NaHCO₃, phosphate) in the dialysis fluid typically also require high amounts of HCl, resulting in non-physiologically high chloride concentrations. Therefore, the concentrations for the buffers should be limited to the lowest possible value.

Preferably, the kit according to the present invention comprises potassium, in particular potassium ions. Too low concentrations of potassium can cause arrhythmia and muscle cramps or paralysis. Patients on the intensive care unit (ICU) can have both, hyperkalemia and hypokalemia. Particular after restoration of an acidosis hypokalemia can occur.

Preferably, the source of potassium, in particular of potassium ions, is KOH and/or KCl, and/or a potassium salt of lactate, acetate, gluconate, citrate, maleate, tartrate and/or of fatty acids such as caprylate. Preferably, in the kit according to the present invention the major source of potassium is KOH and/or KCl.

Preferably, the kit according to the present invention comprises calcium, in particular calcium ions. Too low concentrations of calcium in the patient's blood can cause hypotension or cardiac arrhythmia. Moreover, calcium has a protective effect on the structure of a carrier protein, such as albumin.

In the patient's blood, calcium is present in ionized, protein-bound and complex-like type. The higher the pH value of the dialysis fluid, the more free calcium of the dialysis fluid binds to the carrier protein, such as albumin, comprised by the dialysis fluid. The decreased concentration of ionized calcium in the dialysis fluid triggers a diffusion of free calcium from blood to the dialysate, which may cause decreased calcium levels in the patient.

Preferably, the source of calcium, in particular of calcium ions, is CaCl₂, CaCO₃, and/or a calcium salt of lactate, acetate, gluconate, citrate, maleate, tartrate and/or of fatty acids, preferably the source of calcium is a calcium salt of lactate, acetate, gluconate, citrate, maleate and/or tartrate.

Preferably calcium, in particular calcium ions, is/are not present in the alkaline composition (b).

Preferably, the kit according to the present invention comprises magnesium, in particular magnesium ions. Too low magnesium values in the patient's blood can cause severe cardiac arrhythmias or muscle cramps. Therefore, magnesium is preferably added to the dialysis fluid. Moreover, similar to calcium, magnesium has a protective effect on the structure of a carrier protein, such as albumin. Interestingly, the present inventors have found that the pH in the dialysis fluid affects the magnesium concentration by far less than the calcium concentration.

Preferably, the source of magnesium, in particular of magnesium ions, is MgCl₂, MgCO₃, and/or a magnesium salt of lactate, acetate, gluconate, citrate, maleate, tartrate and/or of fatty acids, preferably the source of magnesium is a magnesium salt of lactate, acetate, gluconate, citrate, maleate and/or tartrate.

Preferably magnesium, in particular magnesium ions, is/are not present in the alkaline composition (b).

In particular if the kit according to the present invention is used on patients on the ICU, the kit preferably comprises phosphate, in particular phosphate ions (H₂PO_{4hu -}, HPO₄²⁻ or PO₄³⁻). For example, in patients on the ICU hypophosphatemia is observed. Therefore, the kit according to the present invention preferably comprises phosphate.

Preferably, the source of phosphate (ions) is a salt of phosphoric acid, in particular any kind of sodium phosphate, potassium phosphate, calcium phosphate and/or magnesium phosphate such as NaH₂PO₄, Na₂HPO₄, Na₃PO₄, KH₂PO₄, K₂HPO₄, K₃PO₄, CaHPO₄, (Ca₃(PO₄)₂), Ca(H₂PO₄)₂, (Ca₅(PO₄)₃.OH), Ca₂P₂O₇, MgHPO₄, Mg₃(PO₄)₂, Mg(H₂PO₄)₂, Mg₂P₂O₇, (Mg₅(PO₄)₃.OH) and combinations thereof. Sodium and potassium salts of phosphoric acid, such as NaH₂PO₄, Na₂HPO₄, Na₃PO₄, KH₂PO₄, K₂HPO₄, K₃PO₄ and combinations thereof are preferred.

Preferably, the kit according to the present invention comprises carbonate/bicarbonate (hydrogen carbonate), in particular (hydrogen) carbonate ions, such as HCO₃⁻ and CO₃²⁻, for example as a bicarbonate buffer system. Carbonate/bicarbonate (hydrogen carbonate) is the main buffering substance used for hemodialysis. In order to replace and to buffer all acids remaining due to a reduced lung, liver or kidney function in the patient, non-physiologically high concentrations of carbonate/bicarbonate (hydrogen carbonate) in the dialysis fluid were necessary in conventional dialysis. However, buffering with HCO₃⁻ also increases CO₂ which may result in an increased cellular acidity in the beginning of a dialysis treatment session. However, too low concentrations of carbonate/bicarbonate (hydrogen carbonate) may be dangerous in patients with metabolic acidosis. The blood buffer capacity of carbonate/bicarbonate (hydrogen carbonate) improves with higher concentrations of carbonate/bicarbonate (hydrogen carbonate).

Preferably, the source of carbonate/bicarbonate (hydrogen carbonate), in particular of (hydrogen) carbonate ions such as CO₃²⁻ and HCO₃⁻, is sodium bicarbonate, sodium carbonate, carbonate, hydrogen carbonate citric acid and/or hydrogen carbonate acetate (the latter two compounds are transformed to bicarbonate in the liver). In general, carbonate/bicarbonate can be added in the form of any of its salts, such as sodium bicarbonate, potassium bicarbonate, and others, or alternatively be added indirectly by introducing carbon dioxide, optionally in the presence of carbonic anhydrase, and adjusting the pH as required by addition of a suitable base, such as sodium hydroxide or potassium hydroxide, sodium hydroxide being strongly preferred. In case of addition in the form of a salt, sodium bicarbonate or sodium carbonate is strongly preferred. Alternatively, potassium salts, or mixtures of sodium and potassium salts, can be used. Salts, which are particularly useful to be added to a dialysis liquid having a high pH, are sodium carbonate or potassium carbonate.

Preferably carbonate/bicarbonate (hydrogen carbonate), in particular (hydrogen) carbonate ions, is/are not present in the acidic composition (a).

Preferably, in the kit according to the present invention, the acidic composition (a) comprises at least one component selected from the group consisting of sodium, chloride, calcium, magnesium, potassium and phosphate. More preferably, in the kit according to the present invention, the acidic composition (a) comprises at least chloride.

Preferably, in the kit according to the present invention, the acidic composition (a) comprises sodium, for example derived from a source as described above. Preferred concentrations of sodium in the acidic composition (a) are no more than 1.0 mol/l, preferably no more than 500 mmol/l, more preferably no more than 300 mmol/l, even more preferably no more than 200 mmol/l and most preferably no more than 150 mmol/l. It is also preferred that concentrations of sodium in the acidic composition (a) are in the range from 0.01 mmol/l to 1.0 mol/l, preferably in the range from 0.05 mmol/l to 500 mmol/l, more preferably in the range from 0.1 mmol/l to 300 mmol/l, even more preferably in the range from 0.5 mmol/l to 200 mmol/l and most preferably in the range from 1.0 mmol/l to 150 mmol/l. However, it is also preferred in the kit according to the present invention, that the acidic composition (a) does not comprise sodium.

Preferably, in the kit according to the present invention, the acidic composition (a) comprises chloride, for example derived from a source as described above. Preferred concentrations of chloride in the acidic composition (a) are no more than 2.0 mol/l, preferably no more than 1.0 mol/l, more preferably no more than 500 mmol/l, even more preferably no more than 300 mmol/l and most preferably no more than 250 mmol/l. It is also preferred that concentrations of chloride in the acidic composition (a) are in the range from 1 mmol/l to 2.0 mol/l, preferably in the range from 10 mmol/lto 1.0 mol/l, more preferably in the range from 50 mmol/lto 500 mmol/l, even more preferably in the range from 100 mmol/l to 300 mmol/l and most preferably in the range from 150 mmol/l to 250 mmol/l.

Preferably, in the kit according to the present invention, the acidic composition (a) comprises calcium, for example derived from a source as described above. Preferred concentrations of calcium in the acidic composition (a) are no more than 5.0 mmol/l, preferably no more than 3.0 mmol/l, more preferably no more than 2.88 mmol/l, even more preferably no more than 2.8 mmol/l and most preferably no more than 2.7 mmol/l. It is furthermore preferred that the concentration of calcium in the acidic composition (a) is at least 2.3 mmol/l, preferably at least 2.4 mmol/l, more preferably at least 2.48 mmol/l, even more preferably at least 2.6 mmol/l, still more preferably at least 2.7 mmol/l and most preferably at least 2.8 mmol/l. It is also preferred that concentrations of calcium in the acidic composition (a) are in the range from 0.1 mmol/l to 50 mmol/l, preferably in the range from 0.5 mmol/l to 20 mmol/l, more preferably in the range from 1.0 mmol/l to 10 mmol/l, even more preferably in the range from 2.0 mmol/l to 5.0 mmol/l and most preferably in the range from 2.3 mmol/l to 3.0 mmol/l. A concentration of calcium in the acidic composition (a) in the range of 2.48-2.88 mmol/l is particularly preferred. Most preferably, the concentration of calcium in the acidic composition (a) is 2.5-2.8 mmol/l, for example 2.6 or 2.7 mmol/l. However, it is also preferred in the kit according to the present invention, that the acidic composition (a) does not comprise calcium.

Preferably, in the kit according to the present invention, the acidic composition (a) comprises magnesium, for example derived from a source as described above. Preferred concentrations of magnesium in the acidic composition (a) are no more than 50 mmol/l, preferably no more than 20 mmol/l, more preferably no more than 10 mmol/l, even more preferably no more than 5 mmol/l and most preferably no more than 2 mmol/l. It is also preferred that concentrations of magnesium in the acidic composition (a) are in the range from 0.005 mmol/l to 50 mmol/l, preferably in the range from 0.01 mmol/l to 20 mmol/l, more preferably in the range from 0.05 mmol/l to 10 mmol/l, even more preferably in the range from 0.1 mmol/l to 5.0 mmol/l and most preferably in the range from 0.5 mmol/l to 2.0 mmol/l. However, it is also preferred in the kit according to the present invention, that the acidic composition (a) does not comprise magnesium.

Preferably, in the kit according to the present invention, the acidic composition (a) comprises potassium, for example derived from a source as described above. Preferred concentrations of potassium in the acidic composition (a) are no more than 200 mmol/l, preferably no more than 100 mmol/l, more preferably no more than 50 mmol/l, even more preferably no more than 20 mmol/l and most preferably no more than 10 mmol/l. It is also preferred that concentrations of potassium in the acidic composition (a) are in the range from 0.01 mmol/l to 200 mmol/l, preferably in the range from 0.05 mmol/l to 100 mmol/l, more preferably in the range from 0.1 mmol/l to 50 mmol/l, even more preferably in the range from 0.5 mmol/l to 20 mmol/l and most preferably in the range from 1.0 mmol/l to 10 mmol/l. However, it is also preferred in the kit according to the present invention, that the acidic composition (a) does not comprise potassium.

Preferably, in the kit according to the present invention, the acidic composition (a) comprises phosphate, in particular phosphate ions (H₂PO₄⁻, HPO₄²⁻ or PO₄³⁻), preferably HPO₄²⁻, for example derived from a source as described above. Preferred concentrations of phosphate in the acidic composition (a) are no more than 50 mmol/l, preferably no more than 20 mmol/l, more preferably no more than 10 mmol/l, even more preferably no more than 5 mmol/l and most preferably no more than 2 mmol/l. It is also preferred that concentrations of phosphate in the acidic composition (a) are in the range from 0.005 mmol/l to 50 mmol/l, preferably in the range from 0.01 mmol/l to 20 mmol/l, more preferably in the range from 0.05 mmol/l to 10 mmol/l, even more preferably in the range from 0.1 mmol/l to 5.0 mmol/l and most preferably in the range from 0.5 mmol/l to 2.0 mmol/l. However, it is also preferred in the kit according to the present invention, that the acidic composition (a) does not comprise phosphate.

Preferably, in the kit according to the present invention, the alkaline composition (b) comprises at least one component selected from the group consisting of sodium, chloride, potassium, phosphate, carbonate/bicarbonate (hydrogen carbonate), and Tris. More preferably, in the kit according to the present invention, the alkaline composition (b) comprises at least sodium and/or potassium, even more preferably, the alkaline composition (b) comprises at least sodium.

Preferably, in the kit according to the present invention, the alkaline composition (b) comprises sodium, for example derived from a source as described above. Preferred concentrations of sodium in the alkaline composition (b) are no more than 2.0 mol/l, preferably no more than 1.0 mol/l, more preferably no more than 750 mmol/l, even more preferably no more than 500 mmol/l and most preferably no more than 300 mmol/l. It is also preferred that concentrations of sodium in the alkaline composition (b) are in the range from 1 mmol/l to 2.0 mol/l, preferably in the range from 5 mmol/l to 1.0 mol/l, more preferably in the range from 10 mmol/l to 750 mmol/l, even more preferably in the range from 50 mmol/l to 500 mmol/l and most preferably in the range from 100 mmol/l to 300 mmol/l.

Preferably, in the kit according to the present invention, the alkaline composition (b) comprises chloride, for example derived from a source as described above. Preferred concentrations of chloride in the alkaline composition (b) are no more than 500 mmol/l, preferably no more than 100 mmol/l, more preferably no more than 50 mmol/l, even more preferably no more than 20 mmol/l and most preferably no more than 10 mmol/l. It is also preferred that concentrations of chloride in the alkaline composition (b) are in the range from 0.05 mmol/l to 500 mmol/l, preferably in the range from 0.1 mmol/l to 100 mmol/l, more preferably in the range from 0.2 mmol/Ito 50 mmol/l, even more preferably in the range from 0.5 mmol/l to 20 mmol/l and most preferably in the range from 1 mmol/l to 10 mmol/l. However, it is also preferred in the kit according to the present invention, that the alkaline composition (b) does not comprise chloride.

Preferably, in the kit according to the present invention, the alkaline composition (b) comprises potassium, for example derived from a source as described above. Preferred concentrations of potassium in the alkaline composition (b) are no more than 500 mmol/l, preferably no more than 100 mmol/l, more preferably no more than 50 mmol/l, even more preferably no more than 20 mmol/l and most preferably no more than 15 mmol/l. It is also preferred that concentrations of potassium in the alkaline composition (b) are in the range from 0.05 mmol/l to 500 mmol/l, preferably in the range from 0.1 mmol/l to 100 mmol/l, more preferably in the range from 0.5 mmol/l to 50 mmol/l, even more preferably in the range from 1 mmol/l to 20 mmol/l and most preferably in the range from 1 mmol/l to 10 mmol/l. However, it is also preferred in the kit according to the present invention, that the alkaline composition (b) does not comprise potassium.

Preferably, in the kit according to the present invention, the alkaline composition (b) comprises phosphate, in particular phosphate ions (H₂PO₄, HPO₄²⁻ or PO₄³⁻), preferably HPO₄²⁻, for example derived from a source as described above. Preferred concentrations of phosphate in the alkaline composition (b) are no more than 50 mmol/l, preferably no more than 20 mmol/l, more preferably no more than 10 mmol/l, even more preferably no more than 5 mmol/l and most preferably no more than 2 mmol/l. It is also preferred that concentrations of phosphate in the alkaline composition (b) are in the range from 0.005 mmol/Ito 50 mmol/l, preferably in the range from 0.01 mmol/l to 20 mmol/l, more preferably in the range from 0.05 mmol/l to 10 mmol/l, even more preferably in the range from 0.1 mmol/l to 5.0 mmol/l and most preferably in the range from 0.5 mmol/l to 2.0 mmol/l. However, it is also preferred in the kit according to the present invention, that the alkaline composition (b) does not comprise phosphate.

Preferably, in the kit according to the present invention, the alkaline composition (b) comprises carbonate/bicarbonate (hydrogen carbonate), such as HCO₃⁻ and CO₃²⁻, for example derived from a source as described above. Preferred concentrations of carbonate/bicarbonate (hydrogen carbonate) in the alkaline composition (b) are no more than 1.0 mol/l, preferably no more than 500 mmol/l, more preferably no more than 200 mmol/l, even more preferably no more than 100 mmol/l and most preferably no more than 80 mmol/l, such as no more than 60 mmol/l. It is also preferred that concentrations of carbonate/bicarbonate (hydrogen carbonate) in the alkaline composition (b) are in the range from 0.1 mmol/l to 1.0 mol/l, preferably from 1 mmol/l to 500 mmol/l, more preferably from 5 mmol/l to 200 mmol/l, even more preferably from 10 mmol/l to 100 mmol/l and most preferably from 50 mmol/l to 60 mmol/l. However, it is also preferred in the kit according to the present invention, that the alkaline composition (b) does not comprise carbonate/bicarbonate (hydrogen carbonate).

Preferably, in the kit according to the present invention, the alkaline composition (b) comprises Tris (Tris(hydroxymethyl)aminomethane ((HOCH₂)₃CNH₂); also referred to as THAM). Preferred concentrations of Tris in the alkaline composition (b) are no more than 1.0 mol/l, preferably no more than 500 mmol/l, more preferably no more than 100 mmol/l, even more preferably no more than 50 mmol/l and most preferably no more than 20 mmol/l, such as no more than 10 mmol/l. It is also preferred that concentrations of Tris in the alkaline composition (b) are in the range from 0.001 mmol/l to 1.0 mol/l, preferably from 0.01 mmol/l to 100 mmol/l, more preferably from 0.1 mmol/l to 50 mmol/l, even more preferably from 0.5 mmol/l to 20 mmol/l and most preferably from 1 mmol/l to 10 mmol/l. However, it is also preferred in the kit according to the present invention, that the alkaline composition (b) does not comprise Tris.

Preferably, the kit according to the present invention comprises
(c3) an electrolyte composition comprising at least one component selected from the group consisting of sodium, chloride, calcium, magnesium, potassium and phosphate, wherein the electrolyte composition (c3) is different from the acidic composition (a) and from the alkaline composition (b).

It is thus preferred that the electrolyte composition (c3) is provided in a spatially separated manner, for example in a container, which comprises the electrolyte composition (c3) (but which container neither comprises the acidic composition (a) nor the alkaline composition (b)).

The electrolyte composition (c3) may be in solid, e.g. powder, in gel, in partially crystalline, in gas phase or in liquid physical condition. Preferably, the electrolyte composition (c3) is a liquid, such as a solution, in particular an aqueous solution, comprising at least one component selected from the group consisting of sodium, chloride, calcium, magnesium, potassium and phosphate as described above.

Preferably, the electrolyte composition (c3) comprises sodium as described above, for example derived from a source as described above.

Preferably, the electrolyte composition (c3) comprises chloride as described above, for example derived from a source as described above.

Preferably, the electrolyte composition (c3) comprises calcium as described above, for example derived from a source as described above.

Preferably, the electrolyte composition (c3) comprises magnesium as described above, for example derived from a source as described above.

Preferably, the electrolyte composition (c3) comprises potassium as described above, for example derived from a source as described above.

Preferably, the electrolyte composition (c3) comprises phosphate, in particular phosphate ions (H₂PO₄₋, HPO₄²⁻ or PO₄³⁻), preferably HPO₄²⁻, as described above, for example derived from a source as described above.

The concentration of each of the components sodium, chloride, calcium, magnesium, potassium and phosphate in the electrolyte composition (c3) may be selected from the concentration of a certain component selected from sodium, chloride, calcium, magnesium, potassium and phosphate as described above for the acidic composition (a) and for the alkaline composition (b). For example, the concentration of sodium in the electrolyte composition (c3) may be selected from the concentration of sodium in the acidic composition (a) as described above and from the concentration of sodium in the alkaline composition (b) as described above. For example, the concentration of chloride in the electrolyte composition (c3) may be selected from the concentration of chloride in the acidic composition (a) as described above and from the concentration of chloride in the alkaline composition (b) as described above. For example, the concentration of calcium in the electrolyte composition (c3) may be selected from the concentration of calcium in the acidic composition (a) as described above. For example, the concentration of magnesium in the electrolyte composition (c3) may be selected from the concentration of magnesium in the acidic composition (a) as described above. For example, the concentration of potassium in the electrolyte composition (c3) may be selected from the concentration of potassium in the acidic composition (a) as described above and from the concentration of potassium in the alkaline composition (b) as described above. For example, the concentration of phosphate, in particular of phosphate ions (H₂PO₄⁻, HPO₄²⁻ or PO₄³⁻), preferably of HPO₄²⁻, in the electrolyte composition (c3) may be selected from the concentration of phosphate, in particular of phosphate ions (H₂PO₄⁻, HPO₄²⁻ or PO₄³⁻), preferably of HPO₄²⁻, in the acidic composition (a) as described above and from the concentration of phosphate, in particular of phosphate ions (H₂PO4⁻, HPO₄²⁻ or PO₄³⁻, preferably of HPO₄²⁻, in the alkaline composition (b) as described above.

Preferably, a kit according to the present invention preferably comprises a stabilizer composition (c1) and an electrolyte composition (c3), wherein the stabilizer composition (c1) and the electrolyte composition (c3) may be the same composition (c7) or distinct compositions. Preferably, the electrolyte composition (c3) is the same as the stabilizer composition (c1). In other words, it is preferred that the kit according to the present invention comprises an electrolyte/stabilizer composition (c7), which comprises both, at least one component selected from the group consisting of sodium, chloride, calcium, magnesium, potassium and phosphate as described above and the stabilizer, in particular caprylate, as described above. Preferably, the electrolyte/stabilizer composition (c7)is provided in a spatially separated manner, for example in a container, which comprises the electrolyte/stabilizer composition (c7) (but which container neither comprises the acidic composition (a) nor the alkaline composition (b)).

Preferably, a kit according to the present invention preferably comprises a nutrient composition (c2) and an electrolyte composition (c3), wherein the nutrient composition (c2) and the electrolyte composition (c3) may be the same composition (c8) or distinct compositions. Preferably, the electrolyte composition (c3) is the same as the nutrient composition (c2). In other words, it is preferred that the kit according to the present invention comprises an electrolyte/nutrient composition (c8), which comprises both, at least one component selected from the group consisting of sodium, chloride, calcium, magnesium, potassium and phosphate as described above and the nutrient, in particular the sugar such as glucose, as described above. Preferably, the electrolyte/nutrient composition (c8) is provided in a spatially separated manner, for example in a container, which comprises the electrolyte/nutrient composition (c8) (but which container neither comprises the acidic composition (a) nor the alkaline composition (b)).

Preferably, the kit according to the present invention comprises a stabilizer composition (c1), a nutrient composition (c2) and an electrolyte composition (c3), wherein the stabilizer composition (c1), the nutrient composition (c2) and the electrolyte composition (c3) may be the same composition (c11) or distinct compositions. Preferably, the electrolyte composition (c3) is the same as the stabilizer composition (c1), which is the same as the nutrient composition (c2). In other words, it is preferred that the kit according to the present invention comprises an electrolyte/stabilizer/nutrient composition (c11), which comprises (i) the nutrient, in particular the sugar such as glucose, as described above, (ii) at least one component selected from the group consisting of sodium, chloride, calcium, magnesium, potassium and phosphate as described above and (iii) the stabilizer, in particular caprylate, as described above.

It is thus preferred that the kit according to the present invention comprises a composition (c11), which comprises (i) a sugar, preferably glucose, (ii) a stabilizer for a carrier protein, in particular a stabilizer for albumin, preferably a caprylate, and (iii) at least one component selected from the group consisting of sodium, chloride, calcium, magnesium, potassium, and phosphate, wherein the composition (c11) is different from the acidic composition (a) and from the alkaline composition (b). Preferably, the composition (c11) is provided in a spatially separated manner, for example in a container, which comprises the composition (c11) (but which container neither comprises the acidic composition (a) nor the alkaline composition (b)).

It is also preferred that the kit according to the present invention comprises
(c4) a buffering composition comprising a buffering agent, in particular carbonate/bicarbonate (hydrogen carbonate),
   wherein the buffering composition (c4) is different from the acidic composition (a) and from the alkaline composition (b).

Preferably, the buffering composition (c4) is provided in a spatially separated manner, for example in a container, which comprises the buffering composition (c4)) (but which container neither comprises the acidic composition (a) nor the alkaline composition (b)).

However, the buffering agent as described below may also be comprised in the alkaline composition (b) instead of providing a separate buffering composition (c4). However, for higher concentrations of the buffering agent up to 40 mmol/l (e.g. of carbonate/bicarbonate) a separate buffering composition (c4) is preferred, whereas concentrations of the buffering agent (e.g. of carbonate/bicarbonate) up to 60 mmol/lare preferably comprised in the alkaline composition (b).

The buffering composition (c4) may be in solid, e.g. powder, in gel, in partially crystalline, in gas phase or in liquid physical condition. Preferably, the buffering composition (c4) is a liquid, such as a solution, in particular an aqueous solution, comprising a buffering agent, in particular carbonate/bicarbonate (hydrogen carbonate).

Preferred buffering agents comprised by the buffering composition (c4) include any one or more of the following: Tris(hydroxymethyl)aminomethane (Tris, THAM); carbonate/bicarbonate; and water-soluble proteins, preferably albumin.

In general, albumin has the capacity to buffer aqueous liquids, and it is thought that certain amino acid residues of albumin (e.g. imidazole group of histidine, thiol group of cysteine) are important (Caironi et al., Blood Transfus., 2009; 7(4): 259-267), and at more elevated pH values, the amino groups of lysine side chains and of the N-termini may contribute to buffering. However, the buffering capacity of albumin has traditionally been exploited in blood (where it occurs naturally in the human or animal body). Bicarbonate is e.g. known to provide physiological pH buffering system. In the buffering composition (c4), as described herein, the buffering capacity of buffering agents such as albumin, carbonate/bicarbonate, or Tris, respectively, may be employed. Optionally, other inorganic or organic buffering agents may be present. Preferably, the buffering agents in the buffering composition (c4) have at least one pKa value in the range from 6.5 to 10, in particular from 7.0 to 9.0. More preferably, two or three of such buffering agents may be employed, each having a pKa value in the range of 7.0 to 9.0. Suitable additional organic buffering agents include proteins, particularly water-soluble proteins, or amino acids, or Tris; and suitable additional inorganic buffering molecules include HPO₄²⁻/H₂PO₄⁻.

Suitable buffering agents to be comprised in the buffering composition (c4) include in particular any one or more of the following: Tris(hydroxymethyl)aminomethane (Tris, THAM); carbonate/bicarbonate; water-soluble proteins, preferably albumin.

Bicarbonate is characterized by an acidity (pKa) of 10.3 (conjugate base: carbonate). Thus, in an aqueous solution containing bicarbonate, carbonate may be present as well, depending on the pH of the solution. For matters of convenience, the expression “carbonate/bicarbonate” is used herein to refer to both bicarbonate and its corresponding base carbonate. “carbonate/bicarbonate concentration” or “(combined) carbonate/bicarbonate concentration”, or the like, refers herein to the total concentration of carbonate and bicarbonate. For example, “20 mmol/l carbonate/bicarbonate” refers to a composition having a 20 mmol/l total concentration of bicarbonate and its corresponding base carbonate. The ratio of bicarbonate to carbonate will typically be dictated by the pH of the composition.

Tris(hydroxymethyl)aminomethane, usually called “Tris”. Tris(hydroxymethyl)aminomethane is also known as “THAM”. Tris is an organic compound with the formula (HOCH₂)₃CNH₂. The acidity (pKa) of Tris is 8.07. Tris is non-toxic and has previously been used to treat acidosis in vivo (e.g. Kallet et al., Am. J. of Resp. and Crit. Care Med. 161: 1149-1153; Hoste et al., J. Nephrol. 18: 303-7.). In an aqueous solution comprising Tris, the corresponding base may be present as well, depending on the pH of the solution. For matters of convenience, the expression “Tris” is used herein to refer to both Tris(hydroxymethyl)aminomethane and its corresponding base, unless the context dictates otherwise. For example, “20 mmol/l Tris” refers to a composition having a 20 mmol/l total concentration of Tris and its corresponding base. The ratio of Tris(hydroxymethyl)aminomethane to its corresponding base will be dictated by the pH of the composition.

A water-soluble protein is suitable as a buffering agent for the purposes of the present invention if it comprises at least one imidazole (histidine side) chain and/or at least one amino group (lysine) side chain and/or at least one sulfhydryl (cysteine) side chain. These side chains typically have pKa values in the range from 7.0 to 11.0. A protein falls under the definition “water-soluble” if at least 10 g/l of the protein is soluble in aqueous solution having a pH within the range of pH 7.4 - 9. A strongly preferred water-soluble protein in the context of the present invention is albumin, as described herein.

Albumin is a preferred water-soluble protein in the context of the present invention. In general, albumin has good buffering capacity in the desired pH range of pH 6.35-11.4, in particular in the pH range from 6.5 to 10, preferably in the pH range from 7.4 to 9, typically, owing to several amino acid side chains with respective pKa values. In particular, albumin can contribute to the buffering capacity by binding carbonate in the form of carbamino groups.

Preferably, the buffering composition (c4) comprises carbonate/bicarbonate (hydrogen carbonate) as described herein, for example derived from a source as described above. For example, the concentration of carbonate/bicarbonate (hydrogen carbonate) in the buffering composition (c4) may be selected from the concentration of carbonate/bicarbonate (hydrogen carbonate) in the alkaline composition (b) as described above. However, too high concentrations of carbonate/bicarbonate are non-physiological and (combined) carbonate/bicarbonate concentrations above 40 mmol/l are not desirable in the dialysis fluid in view of possible side effects. Therefore, it may be desired to avoid the addition of carbonate/bicarbonate to the dialysis fluid and, accordingly, a preferred kit according to the present invention does not comprise carbonate/bicarbonate. The pH range in which bicarbonate can suitably buffer liquids, such as blood is well known in the art, e.g. from biochemistry textbooks.

Preferably, a kit according to the present invention preferably comprises a buffering composition (c4) and an electrolyte composition (c3), wherein the buffering composition (c4) and the electrolyte composition (c3) may be the same composition (c6) or distinct compositions. Preferably, the electrolyte composition (c3) is the same as the buffering composition (c4). In other words, it is preferred that the kit according to the present invention comprises an electrolyte/buffering composition (c6), which comprises both, at least one component selected from the group consisting of sodium, chloride, calcium, magnesium, potassium and phosphate as described above and the buffering agent, in particular carbonate/bicarbonate (hydrogen carbonate), as described above. Preferably, the electrolyte/buffering composition (c6) is provided in a spatially separated manner, for example in a container, which comprises the electrolyte/buffering composition (c6) (but which container neither comprises the acidic composition (a) nor the alkaline composition (b)).

Preferably, a kit according to the present invention preferably comprises a stabilizer composition (c1) and buffering composition (c4), wherein the stabilizer composition (c1) and the buffering composition (c4) may be the same composition (c9) or distinct compositions. Preferably, the buffering composition (c4) is the same as the stabilizer composition (c1). In other words, it is preferred that the kit according to the present invention comprises a buffering/stabilizer composition (c9), which comprises both, the buffering agent, in particular carbonate/bicarbonate (hydrogen carbonate), as described above and the stabilizer, in particular caprylate, as described above. Preferably, the buffering/stabilizer composition (c9) is provided in a spatially separated manner, for example in a container, which comprises the buffering/stabilizer composition (c9) (but which container neither comprises the acidic composition (a) nor the alkaline composition (b)).

Preferably, a kit according to the present invention preferably comprises a nutrient composition (c2) and a buffering composition (c4), wherein the nutrient composition (c2) and the buffering composition (c4) may be the same composition (c10) or distinct compositions. Preferably, the buffering composition (c4) is the same as the nutrient composition (c2). In other words, it is preferred that the kit according to the present invention comprises a buffering/nutrient composition (c10), which comprises both, the buffering agent, in particular carbonate/bicarbonate (hydrogen carbonate), as described above and the nutrient, in particular the sugar such as glucose, as described above. Preferably, the buffering/nutrient composition (c10) is provided in a spatially separated manner, for example in a container, which comprises the buffering/nutrient composition (c10) (but which container neither comprises the acidic composition (a) nor the alkaline composition (b)).

More preferably, a kit according to the present invention preferably comprises a stabilizer composition (c1), a nutrient composition (c2) and buffering composition (c4), wherein the stabilizer composition (c1), the nutrient composition (c2) and the buffering composition (c4) may be the same composition or distinct compositions. Preferably, the buffering composition (c4) is the same as the stabilizer composition (c1) and as the nutrient composition (c2). In other words, it is preferred that the kit according to the present invention comprises a buffering/nutrient/stabilizer composition, which comprises the buffering agent, in particular carbonate/bicarbonate (hydrogen carbonate), as described above, the nutrient, in particular the sugar such as glucose, as described above, and the stabilizer, in particular caprylate, as described above.

More preferably, a kit according to the present invention preferably comprises a stabilizer composition (c1), an electrolyte composition (c3) and buffering composition (c4), wherein the stabilizer composition (c1), the electrolyte composition (c3) and the buffering composition (c4) may be the same composition or distinct compositions. Preferably, the buffering composition (c4) is the same as the stabilizer composition (c1) and as the electrolyte composition (c3). In other words, it is preferred that the kit according to the present invention comprises a buffering/electrolyte/stabilizer composition, which comprises the buffering agent, in particular carbonate/bicarbonate (hydrogen carbonate), as described above, the stabilizer, in particular caprylate, as described above, and at least one component selected from the group consisting of sodium, chloride, calcium, magnesium, potassium and phosphate as described above.

More preferably, a kit according to the present invention preferably comprises a nutrient composition (c2), an electrolyte composition (c3) and buffering composition (c4), wherein the electrolyte composition (c3), the nutrient composition (c2) and the buffering composition (c4) may be the same composition or distinct compositions. Preferably, the buffering composition (c4) is the same as the electrolyte composition (c3) and as the nutrient composition (c2). In other words, it is preferred that the kit according to the present invention comprises a buffering/nutrient/electrolyte composition, which comprises the buffering agent, in particular carbonate/bicarbonate (hydrogen carbonate), as described above, the nutrient, in particular the sugar such as glucose, as described above, and at least one component selected from the group consisting of sodium, chloride, calcium, magnesium, potassium and phosphate as described above.

Even more preferably, a kit according to the present invention preferably comprises a stabilizer composition (c1), a nutrient composition (c2), an electrolyte composition (c3) and a buffering composition (c4), wherein the stabilizer composition (c1), the nutrient composition (c2), the electrolyte composition (c3) and the buffering composition (c4) may be the same composition (c12) or distinct compositions. Preferably, the buffering composition (c4) is the same as the stabilizer composition (c1), which is the same as the nutrient composition (c2), which is the same as the electrolyte composition (c3). In other words, it is preferred that the kit according to the present invention comprises an electrolyte/stabilizer/nutrient/buffering composition (c12), which comprises (i) the nutrient, in particular the sugar such as glucose, as described above, (ii) at least one component selected from the group consisting of sodium, chloride, calcium, magnesium, potassium and phosphate as described above, (iii) the stabilizer, in particular caprylate, as described above and (iv) the buffering agent, in particular carbonate/bicarbonate (hydrogen carbonate), as described above. Preferably, the composition (c12) is provided in a spatially separated manner, for example in a container, which comprises the composition (c12) (but which container neither comprises the acidic composition (a) nor the alkaline composition (b)).

Still more preferably, the kit according to the present invention comprises a composition (c12), which comprises
(i) a sugar, preferably glucose;
(ii) a stabilizer for a carrier protein, in particular a stabilizer for albumin, preferably a caprylate;
(iii) at least one component selected from the group consisting of sodium, chloride, calcium, magnesium, potassium and phosphate and
(iv) a buffering agent, preferably carbonate/bicarbonate (hydrogen carbonate); wherein the composition (c12) is different from the acidic composition (a) and from the alkaline composition (b).

Preferably, the kit according to the present invention comprises
(a) an acidic composition comprising at least one component selected from the group consisting of sodium, chloride, calcium, magnesium, potassium and phosphate, and
(b) an alkaline composition comprising at least one component selected from the group consisting of sodium, chloride, potassium, phosphate, carbonate/bicarbonate (hydrogen carbonate), and Tris, and, optionally, a stabilizer for a carrier protein, in particular a stabilizer for albumin.

Thereby, the acidic composition (a) comprises preferably at least chloride and the alkaline composition (b) comprises preferably at least sodium and/or potassium, more preferably at least sodium. In such a composition, the concentrations of each of the components in the acidic composition (a) may be selected as described above for the concentrations in the acidic composition (a). Accordingly, in such a composition, the concentrations of each of the components in the alkaline composition (b) may be selected as described above for the concentrations in the alkaline composition (b).

It is also preferred that the kit according to the present invention comprises
(a) an acidic composition comprising at least one component selected from the group consisting of sodium, chloride, calcium, magnesium, potassium and phosphate;
(b) an alkaline composition comprising at least one component selected from the group consisting of sodium, chloride, potassium, phosphate and carbonate/bicarbonate (hydrogen carbonate); and
   a stabilizer composition (c1) as described above comprising a stabilizer for a carrier protein, in particular a stabilizer for albumin, such as caprylate, as described above, wherein the stabilizer composition (c1) is different from the acidic composition (a) and from the alkaline composition (b); and/or
   a nutrient composition (c2) as described above comprising a nutrient, in particular a sugar such as glucose, as described above, wherein the nutrient composition (c2) is different from the acidic composition (a) and from the alkaline composition (b); wherein
   if the kit comprise a stabilizer composition (c1) and a nutrient composition (c2), the stabilizer composition (c1) and a nutrient composition (c2) may be the same composition (c5) or distinct compositions.

More preferably, such a kit comprises a stabilizer/nutrient composition (c5), which comprises
a sugar, preferably glucose, and
a stabilizer for a carrier protein, in particular a stabilizer for albumin, preferably a caprylate,
   wherein the composition (c5) is different from the acidic composition (a) and from the alkaline composition (b).

In other words, the kit according to the present invention comprises preferably (i) an acidic composition (a) as described herein, an alkaline composition (b) as described herein and a stabilizer composition (c1) as described herein; (ii) an acidic composition (a) as described herein, an alkaline composition (b) as described herein and a nutrient composition (c2) as described herein; or (iii) an acidic composition (a) as described herein, an alkaline composition (b) as described herein, a stabilizer composition (c1) as described herein and a nutrient composition (c2) as described herein, wherein the latter compositions (c1) and (c2) maybe the same or different compositions, preferably compositions (c1) and (c2) are the same, composition (c5).

Even more preferably, such a kit comprises a stabilizer/nutrient/electrolyte composition (c1 1), which comprises
a sugar, preferably glucose,
a stabilizer for a carrier protein, in particular a stabilizer for albumin, preferably caprylate, and
at least one component selected from the group consisting of sodium, chloride, calcium, magnesium, potassium and phosphate;
   wherein the composition (c11) is different from the acidic composition (a) and from the alkaline composition (b).

Furthermore, the kit—in particular any of the stabilizer composition (c1), the nutrient composition (c2), the electrolyte composition (c3), the buffering composition (c4) and the compositions combined thereof ((c5) to (c12)) as described herein—may comprise additional components such as urea; compounds for diluting blood or inhibiting coagulation and/or platelet aggregation such as heparin or aspirin; and/or fruit acids or salts thereof such as citrate, maleate, tartrate or the like. For example, the advantage of the latter is to reduce the risk of corrosion of the dialysis apparatus.

In a second aspect the present invention provides a kit for treating a carrier protein-containing multiple pass dialysis fluid comprising
(a) an acidic composition comprising a biologically compatible acid, and
(b) an alkaline composition comprising a biologically compatible base,
   wherein the ratio of the concentration of the biologically compatible acid in the acidic composition (a) to the concentration of the biologically compatible base in the alkaline composition (b) is less than 0.8, preferably less than 0.75, more preferably less than 0.7, even more preferably less than 0.675, most preferably less than 0.65, for example about 0.625, and
wherein the concentration of the biologically compatible acid in the acidic composition and the concentration of the biologically compatible base in the alkaline composition is at least 50 mmol/l and no more than 500 mmol/l.

This kit according to the second aspect of the present invention differs from the kit according to the first aspect of the second invention in that the ratio of the concentration of the biologically compatible acid in the acidic composition (a) to the concentration of the biologically compatible base in the alkaline composition (b) is lower. Thus, a dialysis fluid having a higher pH-value, preferably a pH >10, can be obtained and/or regenerated. Apart from that, the kit according to the second aspect of the present invention essentially corresponds to the kit according to the first aspect of the present invention. In particular, preferred embodiments of the kit according to the second aspect of the present invention correspond to preferred embodiments of the kit according to the first aspect of the present invention. An example of a kit according to the second aspect of the present invention is provided herein as “Kit I” of “Example 1” below (which also serves as “comparative example” for the kits according to the first aspect of the present invention).

Uses and Methods of a Kit According to the Present Invention

In a further aspect the present invention provides the use of a kit according to the present invention as described herein for treating, in particular regenerating, a carrier protein-containing multiple pass dialysis fluid, in particular an albumin-containing multiple pass dialysis fluid.

As described above, “treating a carrier protein-containing multiple pass dialysis fluid”, as used herein (i.e. throughout the specification), in general refers to (i) bringing each of the constituents of the kit according to the present invention (e.g. each of the acidic composition (a), the alkaline composition (b), and any further optional constituent such as compositions (c1)-(c12) as described herein) in contact with a carrier protein-containing multiple pass dialysis fluid, thereby (ii) influencing the properties of the carrier protein-containing multiple pass dialysis fluid, for example changing the pH value of the dialysis fluid, changing the composition of the dialysis fluid, changing the density, electrical resistance, conductivity, vapor pressure, viscosity, buffer capacity, surface tension, refractivity, and other constitutive properties of the dialysis fluid, and/or—most preferably—regenerating the carrier protein. In this context, the term “treating a carrier protein-containing multiple pass dialysis fluid”, as used herein (i.e. throughout the specification), refers preferably to regenerating the carrier protein in the carrier protein-containing multiple pass dialysis fluid as described herein. In particular, each of the constituents of the kit according to the present invention (e.g. each of the acidic composition (a), the alkaline composition (b), and any further optional constituent such as compositions (c1)-(c12) as described herein) is directly added to the carrier protein-containing multiple pass dialysis fluid. Preferably, each of the constituents of the kit according to the present invention (e.g. each of the acidic composition (a), the alkaline composition (b), and any further optional constituent such as compositions (c1)-(c12) as described herein) is added to the carrier protein-containing multiple pass dialysis fluid directly in a separate manner. In other words, the constituents of the kit (e.g. the acidic composition (a), the alkaline composition (b), and any further optional constituent such as compositions (c1)-(c12) as described herein) are preferably not mixed with each other before they are brought in contact with (e.g. added to) the carrier-protein-containing multiple pass dialysis fluid.

The term “regenerating” as used herein (i.e. throughout the specification), in particular in the context of “regenerating a carrier protein, such as albumin”, means that after passing the dialyzer substances, which are to be removed from the blood, such as toxins, are bound to the carrier protein. These substances need to be released from the carrier protein in order to reuse the carrier protein in the next cycle of a multiple-pass dialysis. Accordingly, “regenerating” (a carrier protein) means that the carrier protein is transferred from a state (X), in which toxins or other substances to be removed are bound to the carrier protein, to a state (Y), in which the carrier protein is “unbound” (or free). In particular, in such an unbound state (Y) the carrier protein has a conformation enabling the carrier protein to bind to toxins and other substances to be removed from the blood.

A “carrier-protein-containing multiple pass dialysis fluid”, as used herein, refers to a dialysis fluid, which (i) repeatedly (preferably in a continuous or pulsatile manner) passes the dialyzer (and is thus repeatedly used for dialyzing blood) and (ii) comprises a carrier-protein, i.e. a protein, which is involved in the movement of ions, such as protons or hydroxide ions (H⁺ or OH⁻), gases, small molecules or macromolecules. In particular, the carrier protein in the dialysis fluid enables the removal of toxic and/or undesirable ions, such as protons or hydroxide ions (H⁺ or OH⁻), gases, small molecules or macromolecules from the blood during dialysis. The carrier protein is preferably a water-soluble protein. In the context of the present invention as described herein a preferred carrier protein is albumin, preferably serum albumin, more preferably mammalian serum albumin, such as bovine or human serum albumin and even more preferably human serum albumin (HSA). Albumin may be used as it occurs in nature or may be genetically engineered albumin. Mixtures containing albumin and at least one further carrier protein and mixtures of different types of albumin, such as a mixture of human serum albumin and another mammalian serum albumin, are also preferred. In any case, the albumin concentration specified herein refers to the total concentration of albumin, no matter if one single type of albumin (e.g. human serum albumin) or a mixture of various types of albumin is used. The dialysis fluid used in the present invention comprises 3 to 80 g/l albumin, preferably 12 to 60 g/l albumin, more preferably 15 to 50 g/l albumin, and most preferably about 20 g/l albumin. The concentration of albumin can also be indicated as % value and, thus, for example 30 g/l albumin correspond to 3% albumin (wt./vol).

The present invention also provides the use of a kit according to the present invention as described herein for producing (or “generating”) a carrier protein-containing multiple pass dialysis fluid, in particular an albumin-containing multiple pass dialysis fluid.

In a further aspect the present invention provides a method for regenerating a carrier protein-containing multiple pass dialysis fluid, wherein the carrier protein-containing multiple pass dialysis fluid is treated
with an acidic composition (a), which comprises a biologically compatible acid, and
with an alkaline composition (b), which comprises a biologically compatible base, wherein the ratio of the concentration of the biologically compatible acid in the acidic composition (a) to the concentration of the biologically compatible base in the alkaline composition (b) is in the range from 0.7 to 1.3, preferably in the range from 0.75 to 1.25 and more preferably in the range from 0.8 to 1.2 and wherein the concentration of the biologically compatible acid in the acidic composition and the concentration of the biologically compatible base in the alkaline composition is at least 50 mmol/l and no more than 500 mmol/l.

Preferably, the acidic composition (a) as described herein, which is used for treating the carrier protein-containing multiple pass dialysis fluid as described herein, has a pH in the range from 0.5 to 3.0, preferably in the range from 0.7 to 2.0, more preferably in the range from 0.9 to 1.2 and most preferably in the range from 1.0 to 1.1, for example about 1.05. As described above, the carrier protein comprised by the carrier protein-containing multiple pass dialysis fluid unfolds in extremely acidic pH values, thereby releasing the carried substance, e.g. a toxin. The free-floating toxin can then be easily removed, e.g. by filtration. On the other hand, exposure of the carrier protein to an extremely acidic pH value may result in denaturation of the carrier protein. Intensive testing has revealed that a pH value of the dialysis fluid, which is in the range from 1.5 to 5, preferably in the range from 1.8 to 4.5 and more preferably in the range from 2.3 to 4, enables sufficient removal of the toxins and avoids denaturation of the carrier protein. Such a pH value of the dialysis fluid is obtained by addition of an acidic composition (a) having a pH in the range from 0.5 to 3.0, preferably in the range from 0.7 to 2.0, more preferably in the range from 0.9 to 1.2 and most preferably in the range from 1.0 to 1.1, for example about 1.05, to the dialysis fluid (which has a pH in the range from 6.35 to 11.4, in particular from 6.5 to 10, preferably from 7.4 to 9, before adding the acidic composition (a)).

Preferably, the alkaline composition (b) as described herein, which is used for treating the carrier protein-containing multiple pass dialysis fluid as described herein, has a pH in the range from 10.0 to 14.0, preferably in the range from 11.5 to 13.5, more preferably in the range from 12.0 to 13.0 and most preferably in the range from 12.3 to 12.9, for example about 12.6. As described above, the carrier protein comprised by the carrier protein-containing multiple pass dialysis fluid unfolds in extremely alkaline pH values, thereby releasing the carried substance, e.g. a toxin. The free-floating toxin can then be easily removed, e.g. by filtration. On the other hand, exposure of the carrier protein to an extremely alkaline pH value may result in denaturation of the carrier protein. Intensive testing has revealed that a pH value of the dialysis fluid, which is in the range from 9.5 to 12.5, preferably in the range from 10.5 to 12.0 and more preferably in the range from 11 to 11.5, enables sufficient removal of the toxins and avoids denaturation of the carrier protein. Such a pH value of the dialysis fluid is obtained by addition of an alkaline composition (b) having a pH in the range from 10.0 to 14.0, preferably in the range from 11.5 to 13.5, more preferably in the range from 12.0 to 13.0 and most preferably in the range from 12.3 to 12.9, for example about 12.6, to the dialysis fluid (which has a pH in the range from 6.35 to 11.4, in particular from 6.5 to 10, preferably from 7.4 to 9, before adding the alkaline composition (b)).

Preferably, in the method according to the present invention, the treatment of the carrier protein-containing multiple pass dialysis fluid with the acidic composition (a) and with the alkaline composition (b) occurs consecutively. For example, the carrier protein-containing multiple pass dialysis fluid may be treated first with the acidic composition (a) and, thereafter, with the alkaline composition (b). Alternatively, the carrier protein-containing multiple pass dialysis fluid may be treated first with the alkaline composition (b) and, thereafter, with the acidic composition (a). Preferably, such a treatment occurs after the dialysis fluid passed the dialyzer.

However, such a consecutive treatment requires that the pH value of the dialysis fluid is adjusted two times, namely, after the first treatment with the acidic or alkaline composition and after the second treatment with the other (of the acidic or alkaline) composition.

Therefore, it is more preferred, if the method according to the present invention as described herein comprises the following steps:
(i) passing the carrier protein-containing multiple pass dialysis fluid through a dialyzer,
(ii) dividing, i.e. splitting, the flow of the carrier protein-containing multiple pass dialysis fluid, which in particular carries toxins, into a first flow and a second flow,
(iii) adding the acidic composition (a) to the first flow of the carrier protein-containing multiple pass dialysis fluid and the alkaline composition (b) to the second flow of the carrier protein-containing multiple pass dialysis fluid,
(iv) filtration of the first flow of the carrier protein-containing multiple pass dialysis fluid treated with the acidic composition (a) and of the second flow of the carrier protein-containing multiple pass dialysis fluid treated with the alkaline composition (b),
(v) rejoining, i.e. merging, the first flow of the carrier protein-containing multiple pass dialysis fluid treated with the acidic composition (a) and of the second flow of the carrier protein-containing multiple pass dialysis fluid treated with the alkaline composition (b), and
(vi) optionally, performing a further cycle beginning with step (i).

The principle of such a method as well as further details of such a method and a device, which can be used to perform such a method, are described in WO 2009/071103 A1, which is incorporated herein by reference in its entirety.

In such a method it is preferred that, in step the addition of the acidic composition (a) to the first flow of the carrier protein-containing multiple pass dialysis fluid occurs at about the same time as the addition of the alkaline composition (b) to the second flow of the carrier protein-containing multiple pass dialysis fluid.

Moreover, it is preferred in a method according to the present invention, as described herein, that the carrier protein-containing multiple pass dialysis fluid is treated with a stabilizer composition (c1), which comprises a stabilizer for a carrier protein, in particular a stabilizer for albumin, such as caprylate, as described herein. In particular it is preferred that in a method according to the present invention, as described herein, a stabilizer composition (c1) as described herein is (directly) added to the carrier protein-containing multiple pass dialysis fluid.

It is also preferred in a method according to the present invention, as described herein, that the carrier protein-containing multiple pass dialysis fluid is treated with a nutrient composition (c2), which comprises a nutrient, in particular a sugar such as glucose, as described herein. In particular it is preferred that in a method according to the present invention, as described herein, a nutrient composition (c2) as described herein is (directly) added to the carrier protein-containing multiple pass dialysis fluid.

Furthermore it is preferred in a method according to the present invention, as described herein, that the carrier protein-containing multiple pass dialysis fluid is treated with an electrolyte composition (c3), which comprises at least one component selected from the group consisting of sodium, chloride, calcium, magnesium, potassium and phosphate as described herein. In particular it is preferred that in a method according to the present invention, as described herein, an electrolyte composition (c3) as described herein is (directly) added to the carrier protein-containing multiple pass dialysis fluid.

It is also preferred in a method according to the present invention, as described herein, that the carrier protein-containing multiple pass dialysis fluid is treated with a buffering composition (c4), which comprises at least one component selected from the group consisting of sodium, chloride, calcium, magnesium, potassium, phosphate, Tris, protein HSA and carbonate/bicarbonate (hydrogen carbonate) as described herein. In particular it is preferred that in a method according to the present invention, as described herein, a buffering composition (c4) as described herein is (directly) added to the carrier protein-containing multiple pass dialysis fluid.

More preferably, in the method according to the present invention, as described herein, the carrier protein-containing multiple pass dialysis fluid is treated with a stabilizer composition (c1), preferably comprising caprylate as described herein, and a nutrient composition (c2), preferably comprising a sugar such as glucose as described herein, wherein the stabilizer composition (c1) and the nutrient composition (c2) may be the same composition or distinct compositions, preferably the stabilizer composition (c1) and the nutrient composition (c2) are the same composition (c5).

Even more preferably, in the method according to the present invention, as described herein, the carrier protein-containing multiple pass dialysis fluid is treated with a stabilizer composition (c1), preferably comprising caprylate as described herein, a nutrient composition (c2), preferably comprising a sugar such as glucose as described herein, and/or an electrolyte composition (c3), wherein the stabilizer composition (c1), the nutrient composition (c2) and/or the electrolyte composition (c3) may the same composition or distinct compositions, preferably the stabilizer composition (c1), the nutrient composition (c2) and/or the electrolyte composition (c3) are the same composition (c11).

Particularly preferably, in the method according to the present invention, as described herein, the carrier protein-containing multiple pass dialysis fluid is treated with a stabilizer composition (c1), preferably comprising caprylate as described herein, a nutrient composition (c2), preferably comprising a sugar such as glucose as described herein, an electrolyte composition (c3) and/or a buffering composition (c4), wherein the stabilizer composition (c1), the nutrient composition (c2), the electrolyte composition (c3) and/or the buffering composition (c4) may be the same composition or distinct compositions, preferably the stabilizer composition (c1), the nutrient composition (c2), the electrolyte composition (c3) and/or the buffering composition (c4) are the same composition (c12).

It is also preferred in a method according to the present invention, as described herein, that the stabilizer composition (c1), the nutrient composition (c2), the electrolyte composition (c3), the buffering composition (c4) and/or any composition combined thereof (e.g., (c5)-(c12), as described herein, are added to the carrier protein-containing multiple pass dialysis fluid
after the treatment of the carrier protein-containing multiple pass dialysis fluid with the acidic composition (a) and with the alkaline composition (b), preferably after step (v) of the method as described above, and/or
before passing the carrier protein-containing multiple pass dialysis fluid through the dialyzer.

It is also preferred in a method according to the present invention, as described herein, that the stabilizer composition (c1), the nutrient composition (c2), the electrolyte composition (c3), the buffering composition (c4) and/or any composition combined thereof (e.g., (c5)-(c12), as described herein, are added to the carrier protein-containing multiple pass dialysis fluid before the treatment preferably before step (ii).

Preferably, the method according to the present invention, as described herein, comprises a step (v-1) following upon step (v) and preceding step (vi):
(v-1) adding to the carrier protein-containing multiple pass dialysis fluid: (i) a stabilizer composition (c1), which comprises a stabilizer for a carrier protein, in particular a stabilizer for albumin, such as caprylate; (ii) a nutrient composition (c2), which comprises a nutrient, in particular a sugar such as glucose; (iii) an electrolyte composition (c3), which comprises at least one component selected from the group consisting of sodium, chloride, calcium, magnesium, potassium and phosphate; and/or (vi) a buffering composition (c4) comprising a buffering agent, in particular carbonate/bicarbonate;
   wherein the stabilizer composition (c1), the nutrient composition (c2), the electrolyte composition (c3) and/or the buffering composition (c4) are the same composition (c12) or different compositions. Preferably, the stabilizer composition (c1), the nutrient composition (c2), the electrolyte composition (c3) and/or the buffering composition (c4) are the same composition (c12).

In a further aspect, the present invention also provides a method for providing a carrier protein-containing multiple pass dialysis fluid comprising the following steps:
(i) providing an acidic composition (a), which comprises a biologically compatible acid, and an alkaline composition (b), which comprises a biologically compatible base, wherein the ratio of the concentration of the biologically compatible acid in the acidic composition (a) to the concentration of the biologically compatible base in the alkaline composition (b) is in the range from 0.7 to 1.3, preferably in the range from 0.75 to 1.25 and more preferably in the range from 0.8 to 1.2 and wherein the concentration of the biologically compatible acid in the acidic composition and the concentration of the biologically compatible base in the alkaline composition is at least 50 mmol/l and no more than 500 mmol/l,
(ii) merging the acidic composition (a) with the alkaline composition (b), and
(iii) adding a carrier protein, preferably albumin, more preferably human serum albumin (HSA).

Thus, a kit according to the present invention as described herein is not only useful in the treatment of a carrier protein-containing multiple pass dialysis fluid, but advantageously may also serve as a “basis” for providing a carrier protein-containing multiple pass dialysis fluid.

Preferably, only the carrier protein itself needs to be added to provide the carrier protein-containing multiple pass dialysis fluid. Thus, the same components of the kit according to the present invention may—e.g., in the beginning of the procedure—provide the “basis” for the dialysis fluid and—e.g., later in the procedure—the necessary components for regeneration of the dialysis fluid. Advantageously, no further components (except from the carrier protein) are necessary to provide a carrier protein-containing multiple pass dialysis fluid—or, any further components such as nutrients, stabilizers, electrolytes, buffering agents etc. as described herein may be added in a modular manner upon requirement.

Preferably, this method furthermore comprises a step (ii-1), which follows upon step (ii) and precedes step (iii):
(ii-1) adding (i) a stabilizer composition (c1), which comprises a stabilizer for a carrier protein, in particular a stabilizer for albumin, such as caprylate as described herein; (ii) a nutrient composition (c2), which comprises a nutrient, in particular a sugar such as glucose as described herein; and/or (iii) an electrolyte composition (c3), which comprises at least one component selected from the group consisting of sodium, chloride, calcium, magnesium, potassium and phosphate as described herein,
   wherein the stabilizer composition (c1), the nutrient composition (c2) and/or the electrolyte composition (c3) may be the same composition (c11) or distinct compositions.

More preferably, the above described step (ii-1) is as follows:
(ii-1) adding (i) a stabilizer composition (c1), which comprises a stabilizer for a carrier protein, in particular a stabilizer for albumin, such as caprylate as described herein; (ii) a nutrient composition (c2), which comprises a nutrient, in particular a sugar such as glucose as described herein; (iii) an electrolyte composition (c3), which comprises at least one component selected from the group consisting of sodium, chloride, calcium, magnesium, potassium and phosphate as described herein; and/or (iv) a buffering composition (c4), which comprises a buffering agent, in particular carbonate/bicarbonate, as described herein,
   wherein the stabilizer composition (c1), the nutrient composition (c2), the electrolyte composition (c3) and/or the buffering composition (c4) may be the same composition or distinct compositions.

In a further aspect, the present invention also provides the use of a kit according to the present invention as described herein in any of the methods according to the present invention as described herein. In particular, in any of the above methods according to the present invention, it is advantageous to use a kit according to the present invention as described herein.

### BRIEF DESCRIPTION OF THE FIGURES

In the following a brief description of the appended figures will be given. The figures are intended to illustrate the present invention in more detail. However, they are not intended to limit the subject matter of the invention in any way.

FIG. 1 shows a schematic representation of an exemplified dialysis system, which is preferably used for a method for regenerating a carrier protein-containing multiple-pass dialysis fluid according to the present invention.

FIG. 2 shows for Example 3 the detoxification, i.e. the removal of bilirubin (A) and urea (B) from blood achieved with Kit H as described in Example 1 in a method as described in Example 2.

FIG. 3 shows for Example 3 the variation of the pH value of the dialysis fluid (A) as well as the pH value of the blood (B). The thick vertical lines on each graph indicate the change of steps during the experiment (i.e. experimental manipulation of the pH value of the dialysis fluid).

FIG. 4 shows for Example 3 the concentration of sodium in the blood and in the dialysis fluid. The vertical lines on the graph indicate the change of pH value in the dialysate during the experiment as indicated and as described in Example 3.

FIG. 5 shows for Example 3 the concentration of potassium in the blood and in the dialysis fluid. The vertical lines on the graph indicate the change of pH value in the dialysate during the experiment as indicated and as described in Example 3.

FIG. 6 shows for Example 3 the concentration of magnesium in the blood and in the dialysis fluid. The vertical lines on the graph indicate the change of pH value in the dialysate during the experiment as indicated and as described in Example 3.

FIG. 7 shows for Example 3 the concentration of calcium in the blood and in the dialysis fluid. The vertical lines on the graph indicate the change of pH value in the dialysate during the experiment as indicated and as described in Example 3.

FIG. 8 shows for Example 3 the concentration of chloride in the blood and in the dialysis fluid. The vertical lines on the graph indicate the change of pH value in the dialysate during the experiment as indicated and as described in Example 3.

FIG. 9 shows for Example 3 the concentration of phosphate in the blood and in the dialysis fluid. The vertical lines on the graph indicate the change of pH value in the dialysate during the experiment as indicated and as described in Example 3.

FIG. 10 shows for Example 4 the effect of different calcium concentrations, namely, 1.90 mmol/l, 2.06 mmol/l, 2.20 mmol/l, 2.32 mmol/l, 2.48 mmol/l, 2.72 mmol/l and 2.88 mmol/l, in composition (a) for treating a carrier protein-containing multiple pass dialysis fluid at pH 9 (of the dialysis fluid) on the calcium concentration in the blood.

FIG. 11 shows for Example 5 the copper concentration in pmol/l in blood during a dialysis using a kit according to the present invention.

FIG. 12 shows schematically for Example 6 the different steps of the simulation model for measuring turbidity.

FIG. 13 shows for Example 8 the concentration of bilirubin in the blood during a dialysis using kits according to the present invention having different concentrations of a protein stabilizer, namely caprylate.

FIG. 14 shows for Example 9 the concentration of 5-(Hydroxymethyl)-2-furaldehyd (HMF), which derives from dehydration of sugar and, thus, indicates the stability of glucose. (A) Stability of a composition comprising glucose, but no caprylate, at different temperatures as indicated. (B) Stability of a composition

### EXAMPLES

In the following, particular examples illustrating various embodiments and aspects of the invention are presented. However, the present invention shall not to be limited in scope by the specific embodiments described herein. The following preparations and examples are given to enable those skilled in the art to more clearly understand and to practice the present invention. The present invention, however, is not limited in scope by the exemplified embodiments, which are intended as illustrations of single aspects of the invention only, and methods which are functionally equivalent are within the scope of the invention. Indeed, various modifications of the invention in addition to those described herein will become readily apparent to those skilled in the art from the foregoing description, accompanying figures and the examples below. All such modifications fall within the scope of the appended claims.

### Example 1

### Examples of Various Kits According to the First Aspect of the Present Invention

In the following preferred exemplified kits according to the first aspect of the present invention are described. In the following kits, the provided compositions of the exemplified kits, in particular the acidic composition (a), the alkaline composition (b) and, optionally, further compositions as described, can be directly used for providing and/or treating a carrier protein-containing multiple pass dialysis fluid. In other words, in a method according to the present invention the provided compositions of the exemplified kits, in particular the acidic composition (a), the alkaline composition (b) and, optionally, further compositions as described, are directly added (undiluted). In particular, no further composition is required for regeneration and/or provision of the carrier protein-containing multiple pass dialysis fluid.

Kit A

The acidic composition (a) comprising a biologically compatible acid is an aqueous solution with the following components:

| | |
|---|---|
| HCl | 185.0 mmol/l |
| CaCl₂ 2H₂O | 3.2 mmol/l |
| MgCl₂ 6H₂O | 1.4 mmol/l |
| Glucose | 300.0 mg/dl |

The alkaline composition (b) comprising a biologically compatible base is an aqueous solution with the following components:

| | |
|---|---|
| NaOH | 195.0 mmol/l |
| Na₂HPO₄ 2H₂O | 1.0 mmol/l |
| KCl | 6.0 mmol/l |
| Na-caprylate (C₈H₁₅O₂Na) | 300.0 mg/dl |

Kit B

The acidic composition (a) comprising a biologically compatible acid is an aqueous solution with the following components:

| | |
|---|---|
| HCl | 110.0 mmol/l |
| NaCl | 110.0 mmol/l |

The alkaline composition (b) comprising a biologically compatible base is an aqueous solution with the following components:

| | |
|---|---|
| NaOH | 135.0 mmol/l |
| Na₂HPO₄ 2H₂O | 1.0 mmol/l |
| KCl | 6.0 mmol/l |
| Na-caprylate (C₈H₁₅O₂Na) | 5.0 mmol/l |

Kit C

The acidic composition (a) comprising a biologically compatible acid is an aqueous solution with the following components:

| | |
|---|---|
| HCl | 110.0 mmol/l |
| NaCl | 90.0 mmol/l |

The alkaline composition (b) comprising a biologically compatible base is an aqueous solution with the following components:

| | |
|---|---|
| NaOH | 135.0 mmol/l |
| Na₂HPO₄ 2H₂O | 1.0 mmol/l |
| KCl | 6.0 mmol/l |
| Na-caprylate (C₈H₁₅O₂Na) | 1.25 mmol/l |

Kit D

The acidic composition (a) comprising a biologically compatible acid is an aqueous solution with the following components:

| | |
|---|---|
| HCl | 164.0 mmol/l |
| NaCl | 12.0 mmol/l |
| KCl | 7.6 mmol/l |
| Na₂HPO₄ 2H₂O | 1.0 mmol/l |
| MgCl₂ 6H₂O | 1.0 mmol/l |
| CaCl₂ 2H₂O | 1.9 mmol/l |

The alkaline composition (b) comprising a biologically compatible base is an aqueous solution with the following components:

| | |
|---|---|
| NaOH | 160.0 mmol/l |
| Na₂CO₃ | 54.0 mmol/l |

Preferably, kit D comprises, in addition to the acidic composition (a) and to the alkaline composition (b), the a stabilizer/electrolyte composition (c7) with the following component: Na-caprylate (C₈H₁₅O₂Na) 240 mmol/l

More preferably, kit D comprises, in addition to the acidic composition (a) and to the alkaline composition (b), a stabilizer/electrolyte/nutrient composition (c11) with the following components:

| | |
|---|---|
| Na-caprylate (C₈H₁₅O₂Na) | 240 mmol/l |
| Glucose | 40 w/w % |

Kit E

The acidic composition (a) comprising a biologically compatible acid is an aqueous solution with the following components:

| | |
|---|---|
| HCl | 184.0 mmol/l |
| KCl | 7.6 mmol/l |
| Na₂HPO₄ 2H₂O | 1.0 mmol/l |
| MgCl₂ 6H₂O | 1.0 mmol/l |
| CaCl₂ 2H₂O | 2.88 mmol/l |

The alkaline composition (b) comprising a biologically compatible base is an aqueous solution with the following components:

| | | |
|---|---|---|
| NaOH | 176.0 | mmol/l |
| Na₂CO₃ | 51.8 | mmol/l |

Preferably, kit E comprises, in addition to the acidic composition (a) and to the alkaline composition (b), a stabilizer/electrolyte composition (c7) with the following component: Na-caprylate (C₈H₁₅O₂Na) 240 mmol/l

More preferably, kit E comprises, in addition to the acidic composition (a) and to the alkaline composition (b), a stabilizer/electrolyte/nutrient composition (c11) with the following components:

| | | |
|---|---|---|
| Na-caprylate (C₈H₁₅O₂Na) | 240 | mmol/l |
| Glucose | 40 | w/w % |

Kit F

The acidic composition (a) comprising a biologically compatible acid is an aqueous solution with the following components:

| | | |
|---|---|---|
| HCl | 164.0 | mmol/l |
| NaCl | 12.0 | mmol/l |
| KCl | 7.6 | mmol/l |
| Na₂HPO₄ 2H₂O | 1.0 | mmol/l |
| MgCl₂ 6H₂O | 1.0 | mmol/l |
| CaCl₂ 2H₂O | 1.9 | mmol/l |

The alkaline composition (b) comprising a biologically compatible base is an aqueous solution with the following components:

| | | |
|---|---|---|
| NaOH | 160.0 | mmol/l |
| Na₂CO₃ | 54.0 | mmol/l |
| KOH | 10.0 | mmol/l |

Preferably, kit F comprises, in addition to the acidic composition (a) and to the alkaline composition (b), a stabilizer/electrolyte composition (c7) with the following component: Na-caprylate (C₈H₁₅O₂Na) 240 mmol/l

More preferably, kit F comprises, in addition to the acidic composition (a) and to the alkaline composition (b), a stabilizer/electrolyte/nutrient composition (c11) with the following components:

| | | |
|---|---|---|
| Na-caprylate (C₈H₁₅O₂Na) | 240 | mmol/l |
| Glucose | 40 | w/w % |

Kit G

The acidic composition (a) comprising a biologically compatible acid is an aqueous solution with the following components:

| | | |
|---|---|---|
| HCl | 164.0 | mmol/l |
| NaCl | 12.0 | mmol/l |
| KCl | 7.6 | mmol/l |
| Na₂HPO₄ 2H₂O | 1.0 | mmol/l |
| MgCl₂ 6H₂O | 1.0 | mmol/l |

The alkaline composition (b) comprising a biologically compatible base is an aqueous solution with the following components:

| | | |
|---|---|---|
| NaOH | 160.0 | mmol/l |
| Na₂CO₃ | 54.0 | mmol/l |
| KOH | 10.0 | mmol/l |

Preferably, kit G comprises, in addition to the acidic composition (a) and to the alkaline composition (b), a stabilizer/electrolyte composition (c7) with the following component: Na-caprylate (C₈H₁₅O₂Na) 240 mmol/l

More preferably, kit G comprises, in addition to the acidic composition (a) and to the alkaline composition (b), a stabilizer/electrolyte/nutrient composition (c11) with the following components:

| | | |
|---|---|---|
| Na-caprylate (C₈H₁₅O₂Na) | 240 | mmol/l |
| Glucose | 40 | w/w % |

Kit H

The acidic composition (a) comprising a biologically compatible acid is an aqueous solution with the following components:

| | | |
|---|---|---|
| HCl | 184.0 | mmol/l |
| NaCl | 6.0 | mmol/l |
| Na₂HPO₄ 2H₂O | 1.0 | mmol/l |
| MgCl₂ 6H₂O | 1.0 | mmol/l |
| Na₃ citrate | 0.8 | mmol/l |
| CaCl₂ 2H₂O | 2.88 | mmol/l |

The alkaline composition (b) comprising a biologically compatible base is an aqueous solution with the following components:

| | | |
|---|---|---|
| NaOH | 168.4 | mmol/l |
| Na₂CO₃ | 51.8 | mmol/l |
| KOH | 7.6 | mmol/l |

Preferably, kit H comprises, in addition to the acidic composition (a) and to the alkaline composition (b), a stabilizer/electrolyte composition (c7) with the following component:

| | |
|---|---|
| Na-caprylate (C₈H₁₅O₂Na) | 240 mmol/l |

More preferably, kit H comprises, in addition to the acidic composition (a) and to the alkaline composition (b), a stabilizer/electrolyte/nutrient composition (c11) with the following components:

| | | |
|---|---|---|
| Na-caprylate (C₈H₁₅O₂Na) | 240 | mmol/l |
| Glucose | 40 | w/w % |

Each of the above kits A-H can be used to obtain/regenerate a carrier protein-containing multiple pass dialysis fluid having a pH from 6.5 to 10, in particular from 7.45 to 9. Kits B and C, which do not comprise calcium, magnesium and bicarbonate, can even be used to obtain/regenerate a carrier protein-containing multiple pass dialysis fluid having a pH from 6.35 to 11.4.

### Comparative Example—Kit I

The acidic composition (a) comprising a biologically compatible acid is an aqueous solution with the following components:

| | | |
|---|---|---|
| HCl | 100.0 | mmol/l |
| NaCl | 12.0 | mmol/l |
| KCl | 7.6 | mmol/l |
| Na₂HPO₄ 2H₂O | 1.0 | mmol/l |

The alkaline composition (b) comprising a biologically compatible base is an aqueous solution with the following components:

| | | |
|---|---|---|
| NaOH | 160.0 | mmol/l |
| NaCl | 68.0 | mmol/l |

Kit I differs from kits A-H primarily in that the ratio of the concentration of the biologically compatible acid in the acidic composition (a) to the concentration of the biologically compatible base in the alkaline composition (b) is 0.625, whereas that ratio is in the range of 0.7 to 1.3 for kits A-H. The carrier protein-containing multiple pass dialysis fluid obtained/regenerated by kit I has a pH>10, whereas with kits A-H the pH of the dialysis fluid can be adjusted to values from 6.5 to 10, in particular from 7.45 to 9.

### Example 2

### Method for Regenerating a Carrier Protein-Containing Multiple-Pass Dialysis Fluid

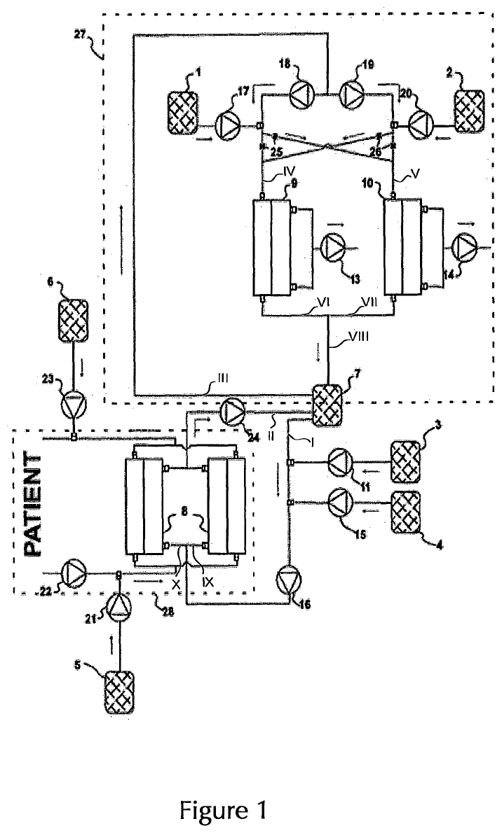

FIG. 1 shows a diagrammatic representation of an exemplified dialysis system, which is preferably used for a method for regenerating a carrier protein-containing multiple-pass dialysis fluid according to the present invention. The dialysis system is described in more detail in WO 2009/071103 A1, which is incorporated herein by reference.

Blood from the patient is transported through the tubings via a blood pump (22). Before it is returned to the patient, the blood is passed through two dialyzers (8) which contain the semipermeable membranes. In said dialyzers the blood is separated from the dialysis fluid by means of the semipermeable membranes. Dilution fluids, namely, predilution (5) and postdilution fluids (6) can be optionally added to the patient's blood via the predilution pump (21) and the postdilution pump (23). Blood flow rates are, in general, between 50-2000 ml/min, typically depending on the type and duration of dialysis. Preferably, blood flow rates are between 150-600 ml/min and more preferably between 250-400 ml/min. Predilution flow rates are preferably between 1-10 l/h and more preferably 4-7 l/h. Postdilution flow rates are preferably between 5-30% of the chosen blood flow rates and more preferably between 15-20%.

The dialysis fluid is pumped into the dialysate compartment of the dialyzers with a pump (16) from the dialysis fluid reservoir (7) at a flow rate between 50-4000 ml/min, preferably between 150-2000 ml/min, more preferably between 500-1100 ml/min and most preferably at about 800 ml/min. The dialysis fluid with the optionally added predilution and postdilution and other fluids taken from the patient to reduce his volume overload are transported back to the dialysis fluid reservoir (7) via a pump (24) at flow rates depending on the flow rates of the predilution, postdilution and the dialysate and the amount of fluid that should be removed from the patient.

In general, the dialysis fluid is cleaned continuously or intermittently by (i) manipulation of the pH and temperature as well as (ii) optically, by irradiating with waves, light, electrical and/or magnetic fields, in combination with addition of further components, such as a stabilizer, a nutrient, a buffer and/or an electrolyte and filtration. After passing through the dialyzer (8) and through the dialysis fluid reservoir (7), the flow of the carrier protein-containing multiple pass dialysis fluid, which contains for example toxins, is split into a first flow and a second flow. The regeneration pumps (18, 19) transport the first flow of the carrier protein-containing multiple pass dialysis fluid and the second flow of the carrier protein-containing multiple pass dialysis fluid through the tubings from and to the dialysis fluid reservoir (7). The pump on the “acid side” (18) and the pump on the “base side” (19) transport the dialysis fluid downstream to one of two filters (9, 10) present in the dialysate regeneration circuit (27) through a valve mechanism (25, 26).

The acidic composition (a), which is stored and/or mixed in a container (1), is added to the first flow of the carrier protein-containing multiple pass dialysis fluid at the “acid side” via a pump (17). The alkaline composition (b), which is stored and/or mixed in a container (2), is added to the the second flow of the carrier protein-containing multiple pass dialysis fluid at the “base side” via a pump (20). Addition of the acidic composition (a)—as well as addition of the alkaline composition (b)—results in a release of the carrier protein-bound toxins from the carrier protein, such as albumin.

The valves (25,26) enable (i) that the first flow of the carrier protein-containing multiple pass dialysis fluid treated with the acidic composition (a) is transported either towards the filter (9) or towards the filter (10) (valve 25) and (ii) that the second flow of the carrier protein-containing multiple pass dialysis fluid treated with the alkaline composition (b) is transported either towards the filter (9) or towards the filter (10) (valve 26). The valves (25, 26) may change the direction of flow for example every 5 min-1 hour, preferably every 10 min, so that each filter (9, 10) receives fluid from one pump (1 8 or 19) at a time.

The first flow of the carrier protein-containing multiple pass dialysis fluid treated with the acidic composition (a) and the second flow of the carrier protein-containing multiple pass dialysis fluid treated with the alkaline composition (b) are filtered in filters (9, 10), thereby removing the toxins and “cleaning” the carrier protein-containing multiple pass dialysis fluid, and fluids are removed from each filter (9, 10) using two filtrate pumps (13, 14). After filtration, the first flow of the carrier protein-containing multiple pass dialysis fluid treated with the acidic composition (a) is rejoined with the second flow of the carrier protein-containing multiple pass dialysis fluid treated with the alkaline composition (b), thereby mixing the first and the second flow.

Optionally, after rejoining the first and the second flow of the carrier protein-containing multiple pass dialysis fluid, a stabilizer composition, a nutrient composition, a buffer composition and/or an electrolyte composition is added thereto. For example, the stabilizer composition, the nutrient composition, the buffer composition and/or the electrolyte composition can be stored and/or diluted in the containers (3, 4) and added to the carrier protein-containing multiple pass dialysis fluid via one or two pumps (11, 15). In more general, the stabilizer composition, the nutrient composition, the buffer composition and/or the electrolyte composition can be preferably added to the dialysis fluid at any of positions I to X shown in FIG. 1.

### Example 3

### Test of Kit H in a Method as Described in Example 2

Kit H as described in Example 1 was tested in a method as described in Example 2 in order to evaluate detoxification and electrolyte content in blood and dialysis fluid at different pH values and flow rates of the dialysis fluid.

To this end, a total of six experiments were performed using porcine blood and two dialysis devices LK2001 (Hepa Wash GmbH, Munich, Germany). The values shown were measured in blood and dialysate, respectively, just before the blood (or the dialysate) entered into the dialyzer. Results are expressed as average of the data of the six experiments.

In order to assess different pH values and flow rates the steps shown in Table 1 below were performed:

TABLE 1

| Duration (hh:mm) | Tested parameters |
|---|---|
| 00:00 to 01:20 | Flow of compostion (a): 160 ml/min |
| | Flow of compostion (b): 160 ml/min |
| | Dialysate pH: 7.45 |
| 01:20 to 02:40 | Flow of compostion (a): 160 ml/min |
| | Flow of compostion (b): 160 ml/min |
| | Dialysate pH: 9/CO₂ 4.8 mmol/min |
| 02:40 to 04:00 | Flow of compostion (a): 80 ml/min |
| | Flow of compostion (b): 80 ml/min |
| | Dialysate pH: 7.45 |
| 04:00 to 05:20 | Flow of compostion (a): 80 ml/min |
| | Flow of compostion (b): 80 ml/min |
| | Dialysate pH: 9/CO₂ 4.8 mmol/min |

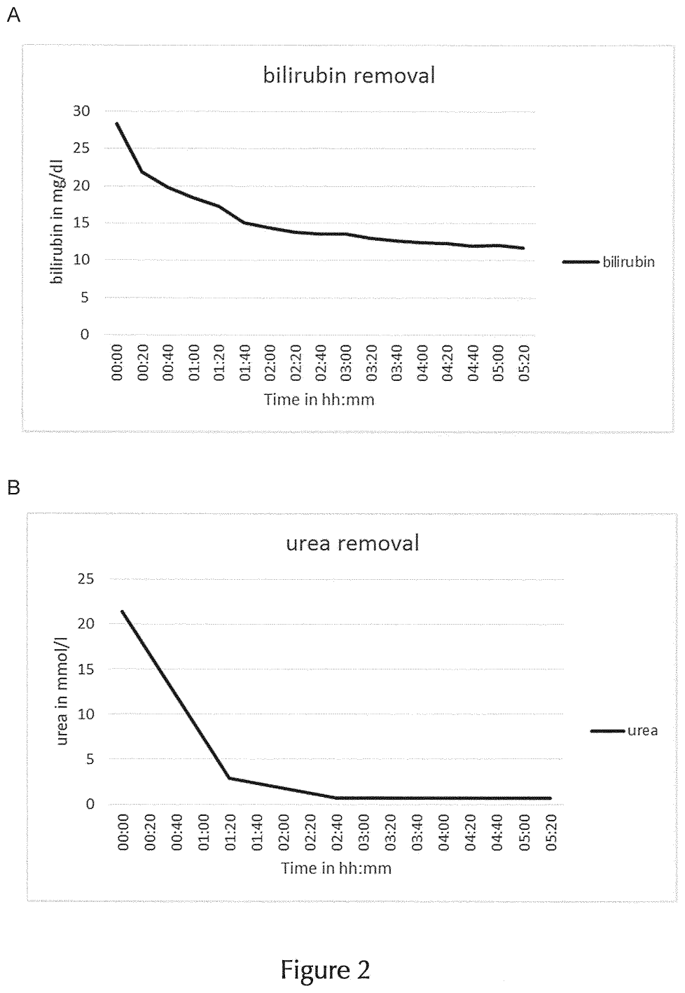

FIG. 2 shows the detoxification (blood bilirubin and urea) achieved in this study. As can be retrieved from FIG. 2, urea levels decrease from more than 20 mmol/l to almost 0 mmol/l (FIG. 2B) and bilirubin levels decrease from almost 30 mg/dl to about 11 mg/dl (FIG. 2A).

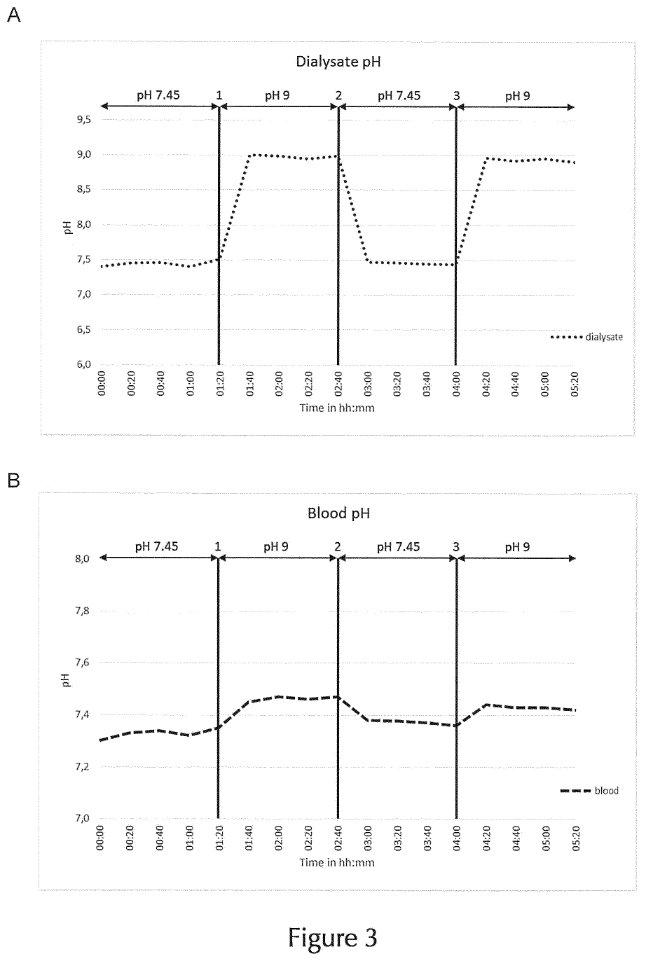

FIG. 3 shows the variation of the pH value of the dialysis fluid (A) as well as the pH value of the blood (B). The thick vertical lines on each graph indicate the change of steps during the experiment as described above (i.e. experimental manipulation of the pH value of the dialysis fluid). As shown in FIG. 3B, the blood pH is raising between 01:20 and 02:40 and between 04:00 and at the end due to the applied dialysate pH of 9 and the adjusted buffering capacity of the dialysate fluid. To simulate a acidosis in the blood, CO₂ was administered to the blood and a dialysate pH of 9 was applied, since acidosis is treated by a dialysis liquid with a pH of 9.

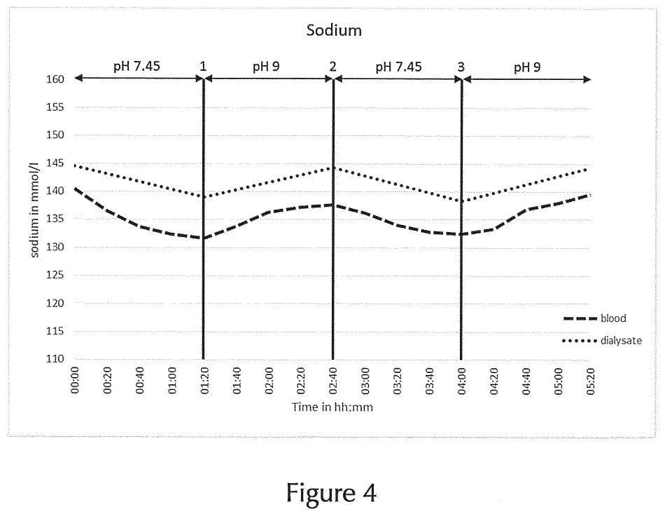

FIG. 4 shows the variation of the sodium concentration in the blood and in the dialysis fluid. The sodium concentrations in the blood are within the physiological limitations of 125-142 mmol/l during the whole treatment. An elevation of the sodium concentration was noted at pH 9.

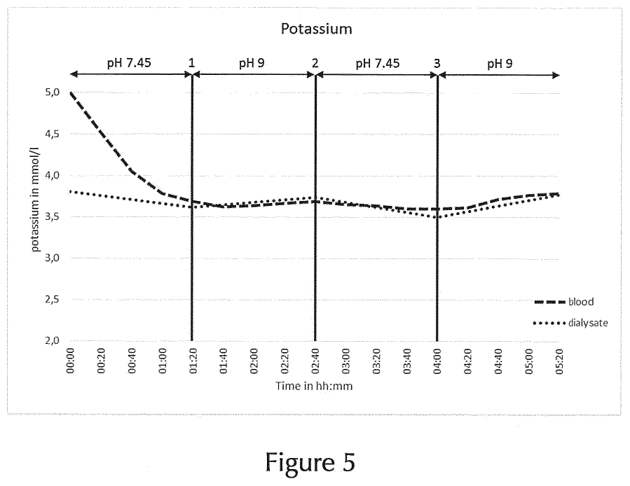

FIG. 5 shows the variation of the potassium concentration in the blood and in the dialysis fluid. The potassium concentrations in the blood are within the physiological limitations of 3.4-4.5 mmol/l. There are no significant changes between dialysate-pH 7.45 and dialysate-pH 9. The first potassium value in the blood is at the border of the range since porcine blood usually shows a high concentration of potassium at the very beginning of the measurement. The dialysate values are also within their limitations of 0-5.0 mmol/l.

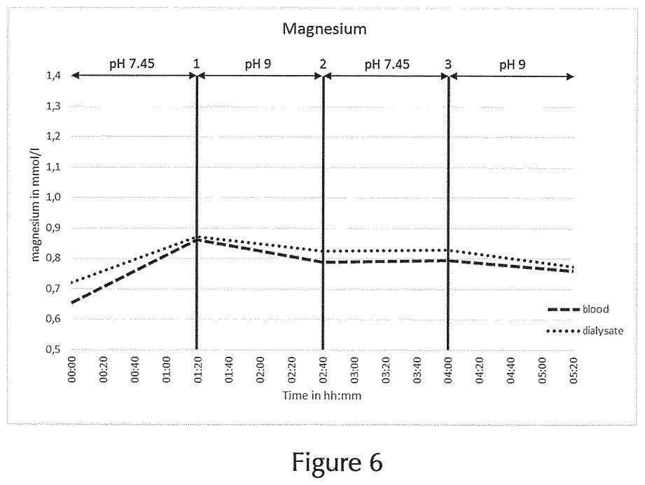

FIG. 6 shows the variation of the magnesium concentration in the blood and in the dialysis fluid. The magnesium concentrations in the blood are within the physiological limitations of 0.5-1.3 mmol/l during the whole treatment. The dialysate values are also within their limitations.

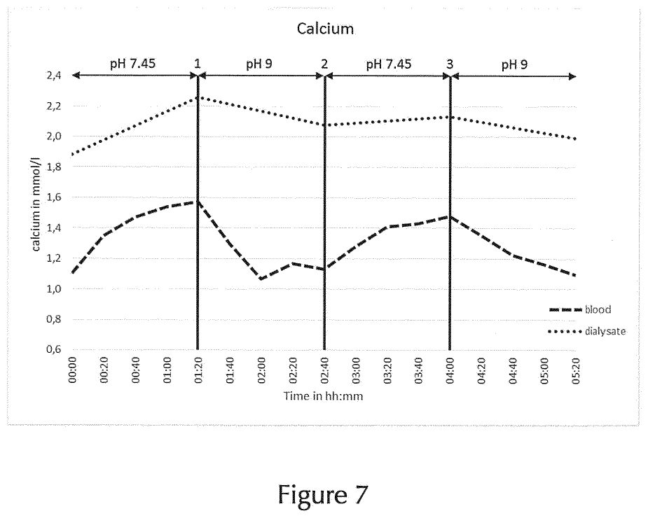

FIG. 7 shows the variation of the calcium concentration in the blood and in the dialysis fluid. The calcium concentrations in the blood are within the physiological limitations of 1.0-1.7 mmol/l during the whole treatment. The dialysate pH of 9 is causing a decrease in the calcium concentration. The dialysate values are also within their limitations.

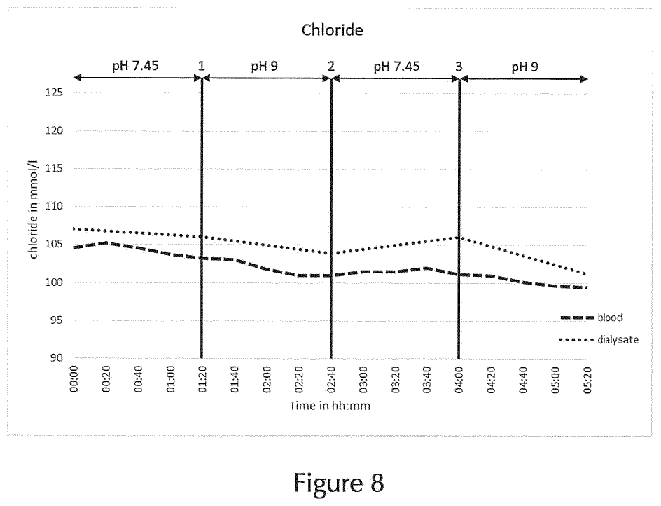

FIG. 8 shows the variation of the chloride concentration in the blood and in the dialysis fluid. The chloride concentrations in the blood are within the physiological limitations of 95-110 mmol/l during the whole treatment. The dialysate values are also within their limitations.

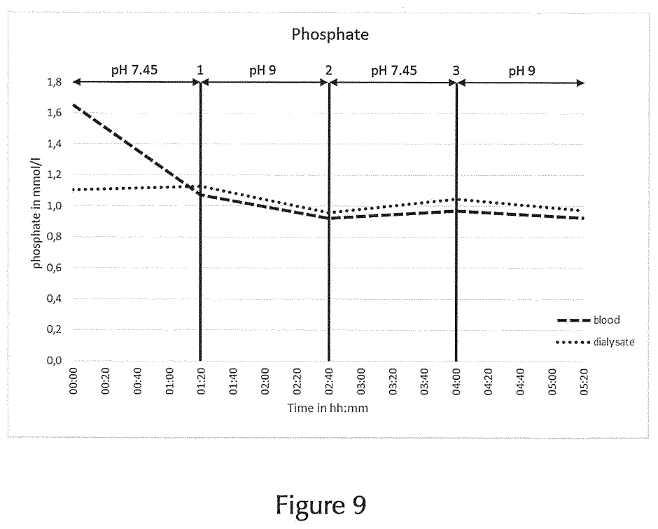

FIG. 9 shows the variation of the phosphate concentration in the blood and in the dialysis fluid. The phosphate concentrations in the blood are within the physiological limitations of 0.5-2 mmol/l during the whole treatment. The dialysate values are also within their limitations.

Taken together, all measured blood concentrations of the electrolytes are within their physiological limitations and the detoxification of the blood was observed. Accordingly, Kit H is useful at varying pH values of the dialysis fluid (7.45 and 9) and at different flow rates of the dialysis fluid.

### Example 4

### Influence of the Dialysate pH on the Calcium Concentration in the Blood

As shown in Example 3 (FIG. 7), the increased dialysate pH of 9 is causing a decrease in the calcium concentration of the blood. Calcium is present in ionized, protein-bound and complex-like type. The higher the pH value of the dialysate, the more free calcium of the dialysis fluid binds to the carrier protein, such as albumin, comprised by the dialysis fluid. The decreased concentration of ionized calcium in the dialysis fluid triggers a diffusion of free calcium from blood to the dialysate, which causes decreased calcium levels in the patient.

Therefore, the influence of the dialysate pH on the calcium concentration in the blood was further investigated in order to provide a kit, which ensures a physiological calcium level in the blood despite treatment with varying pH values of the dialysate.

To this end, experiments were performed using porcine blood and the dialysis device LK2001 (Hepa Wash GmbH, Munich, Germany). In this experiment, the following kits, which differed only in the concentration of CaCl₂, were used:

Kit 4A The acidic composition (a) comprising a biologically compatible acid is an aqueous solution with the following components:

| HCl | 164.0 mmol/l |
|---|---|
| NaCl | 12.0 mmol/l |
| KCl | 7.6 mmol/l |
| Na₂HPO₄ 2H₂O | 1.0 mmol/l |
| MgCl₂ 6H₂O | 1.0 mmol/l |
| CaCl₂ 2H₂O | 1.9 mmol/l |
| pH | 1.05 |

The alkaline composition (b) comprising a biologically compatible base is an aqueous solution with the following components:

| NaOH | 160.0 mmol/l |
|---|---|
| Na₂CO₃ | 54.0 mmol/l |
| pH | 12.6 |

Kit 4B

In kit 4B exactly the same components as in kit 4A were used, except that the concentration of CaCl₂ was 2.06 mmol/l instead of 1.9 mmol/l. Accordingly, kit 4B differed only in the concentration of CaCl₂ from kit 4A.

Kit 4C

In kit 4C exactly the same components as in kit 4A were used, except that the concentration of CaCl₂ was 2.2 mmol/l instead of 1.9 mmol/l. Accordingly, kit 4C differed only in the concentration of CaCl₂ from kit 4A.

Kit 4D

In kit 4D exactly the same components as in kit 4A were used, except that the concentration of CaCl₂ was 2.32 mmol/l instead of 1.9 mmol/l. Accordingly, kit 4D differed only in the concentration of CaCl₂ from kit 4A.

Kit 4E

In kit 4E exactly the same components as in kit 4A were used, except that the concentration of CaCl₂ was 2.48 mmol/l instead of 1.9 mmol/l. Accordingly, kit 4E differed only in the concentration of CaCl₂ from kit 4A.

Kit 4F

In kit 4F exactly the same components as in kit 4A were used, except that the concentration of CaCl₂ was 2.72 mmol/l instead of 1.9 mmol/l. Accordingly, kit 4F differed only in the concentration of CaCl₂ from kit 4A.

Kit 4G

In kit 4G exactly the same components as in kit 4A were used, except that the concentration of CaCl₂ was 2.88 instead of 1.9 mmol/l. Accordingly, kit 4G differed only in the concentration of CaCl₂ from kit 4A.

The acidic composition (a) and the alkaline composition (b) of those kits were used to directly treat the carrier protein-containing multiple pass dialysis fluid.

Preferably, in all of the above kits 4A-4G a stabilizer/nutrient composition (c5) with the following components:

| | |
|---|---|
| Na-caprylate (C₈H₁₅O₂Na) | 240 mmol/l |
| Glucose | 40 w/w % |

was used in addition to the acidic composition (a) and to the alkaline composition (b).

In the above kits, calcium was provided in the acidic composition (a). The source of calcium was CaCl₂. Different acidic compositions (a) were provided, which differed in the calcium concentration. Acidic compositions (a) having the following calcium concentrations were provided in a kit as described above:

1.9 mmol/l, 2.06 mmol/l, 2.2 mmol/l, 2.32 mmol/l, 2.48 mmol/l, 2.72 mmol/l and 2.88 mmol/l, respectively.

These different calcium concentrations were tested in a kit as described above, at a pH value of the dialysis fluid of 9.

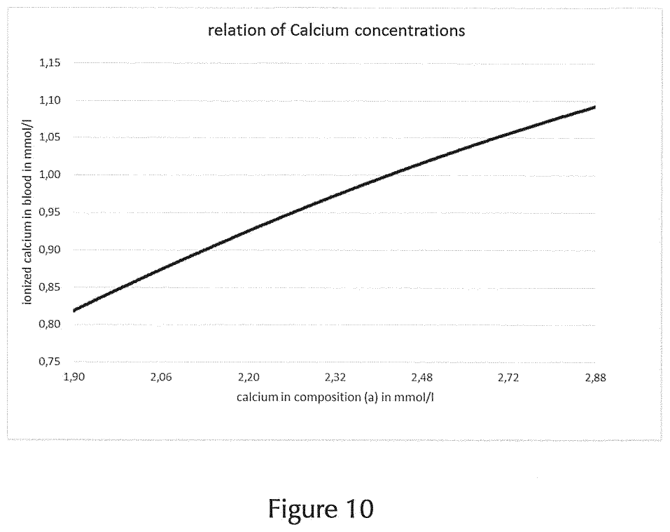

The results are shown in FIG. 10. These results show that a calcium concentration of at least 2.48 mmol/l was necessary to obtain a value of ionized calcium above 1.0 mmol/l in the blood. A calcium concentration of at least 2.88 mmol/l was necessary to obtain a calcium level of about 1.1 mmol/l in the blood.

In the next step, the effects of the highest calcium concentration (2.88 mmol/l) was evaluated at a pH of 7.45 of the dialysis fluid. Under such conditions, a calcium level of 1.7 mmol/l was observed in the blood. Since a physiological calcium level in the blood is in the range from 1.0-1.7 mmol/l, the highest calcium concentration (2.88 mmol/l) in the acidic composition (a) still resulted in a physiological calcium level in the blood.

### Example 5

### Removal of Copper from Blood Using a Kit According to the Present Invention

To assess the ability of the kit according to the present invention to remove copper from blood, experiments were performed using porcine blood and the dialysis device LK2001 (Hepa Wash GmbH, Munich, Germany). In this experiment, a kit comprising the following compositions was used:

The acidic composition (a) comprising a biologically compatible acid is an aqueous solution with the following components:

| HCl | 164.0 mmol/l |
|---|---|
| NaCl | 12.0 mmol/l |
| KCl | 7.6 mmol/l |
| Na₂HPO₄ 2H₂O | 1.0 mmol/l |
| MgCl₂ 6H₂O | 1.0 mmol/l |
| CaCl₂ 2H₂O | 1.9 mmol/l |
| pH | 1.05 |

The alkaline composition (b) comprising a biologically compatible base is an aqueous solution with the following components:

| NaOH | 160.0 mmol/l |
|---|---|
| Na₂CO₃ | 54.0 mmol/l |
| pH | 12.6 |

More preferably, in addition to the acidic composition (a) and to the alkaline composition (b), a stabilizer/nutrient composition (c5) with the following components:

| | |
|---|---|
| Na-caprylate (C₈H₁₅O₂Na) | 240 mmol/l |
| Glucose | 40 w/w % |

The acidic composition (a) and the alkaline composition (b) of this kit were used to directly treat the carrier protein-containing multiple pass dialysis fluid.

Porcine blood was treated for 2 h in the dialysis device LK2001 (Hepa Wash GmbH, Munich, Germany) as described in Example 2 and the concentration of copper in the blood was measured.

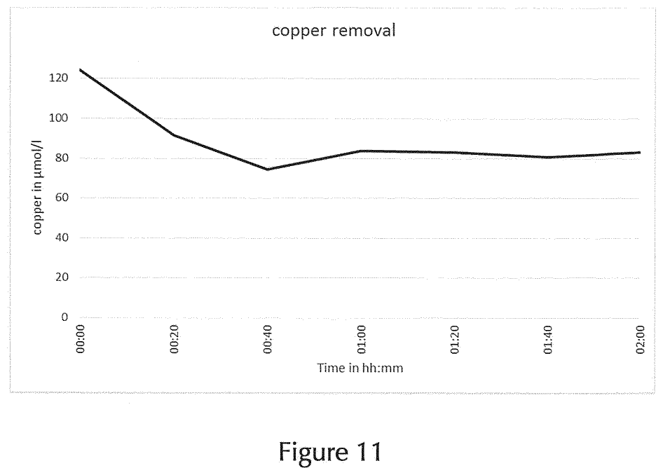

Results are shown in FIG. 11. In about 40 minutes the concentration of copper was reduced from 124.20 μmol/l to 74.40 μmol/l. In other words, more than 40 percent of copper were removed during dialysis.

### Example 6

### Influence of Distinct Protein Stabilizers on the Stability of Albumin

To assess the influence of distinct protein stabilizers on the stability of albumin in a method as described in Example 2, a simulation model for the “neutralization zone” was developed. The term “neutralization zone”, as used herein, refers to that zone in the dialysis apparatus, where the mixing of the first flow of the carrier protein-containing multiple pass dialysis fluid treated with the acidic composition (a) with the second flow of the carrier protein-containing multiple pass dialysis fluid treated with the alkaline composition (b) occurs after their separation, as described in Example 2. In the schematic representation of the exemplified dialysis system shown FIG. 1, the neutralization zone is referred to as “VIII”. In the method of Example 2, the neutralization zone is the zone, in which the carrier protein, such as albumin, is particularly prone to degradation.

Preparation of the Dialysis Fluid (Dialysate)

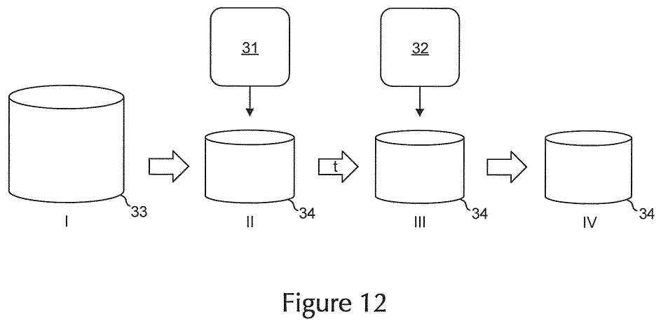

To test the stability of albumin in this simulation model, the solutions of dialysate were freshly prepared before the beginning of every experiment. The dialysate may be prepared in a large canister (33), for example as shown in FIG. 12. To prepare the dialysate the following acidic composition (a) and the alkaline composition (b) were used:

The acidic composition (a) comprising a biologically compatible acid is an aqueous solution with the following components:

| HCl | 164.0 mmol/l |
|---|---|
| NaCl | 12.0 mmol/l |
| KCl | 7.6 mmol/l |
| Na₂HPO₄ 2H₂O | 1.0 mmol/l |
| MgCl₂ 6H₂O | 1.0 mmol/l |
| CaCl₂ 2H₂O | 1.9 mmol/l |
| pH | 1.05 |

The alkaline composition (b) comprising a biologically compatible base is an aqueous solution with the following components:

| NaOH | 160.0 mmol/l |
|---|---|
| Na₂CO₃ | 54.0 mmol/l |
| pH | 12.6 |

To prepare the dialysate the acidic composition (a) and the alkaline composition (b) were mixed and (osmosis) water was added to obtain the concentrations shown in table 2.

The dialysis fluid used had a pH of 7.45 and comprised the components as shown in Table 2 below:

TABLE 2

| | | |
|---|---|---|
| Na⁺ | 138.00 | mmol/l |
| K⁺ | 2.50 | mmol/l |
| Ca²⁺ | 1.50 | mmol/l |
| Mg²⁺ | 0.50 | mmol/l |
| Cl⁻ | 110.00 | mmol/l |
| HCO₃⁻ | 32.00 | mmol/l |
| Glucose | 1.00 | g/l |
| Albumin | 30.00 | g/l |

The concentration of electrolytes was controlled such that it was comparable with the physiological values in the human body and in order to obtain constant conditions in the fluid.

The dialysate as shown in table 2 was then filled into several smaller canisters (34), as shown in FIG. 12. To each of canisters (34), a distinct type and/or concentration of stabilizers was added, as shown in Table 3, and mixed. Therefore, each of the canisters (34) contained the same dialysate (as described in table 2), but differed in the type of stabilizer and/or concentration of the stabilizer, as shown in table 3.

Experiments 01 - 24

All experiments 01 to 24 were performed according to the detailed description of the Stabilization test below. The solutions used and test steps were the same for all experiments but differed only in the stabilizer composition (c1) comprising different stabilizers according to table 3. The stabilizer concentration shown in table 3 is the concentration of each stabilizer in the dialysate in each of the canisters (34), respectively.

In experiments 01-23 different stabilizers were tested. In experiment 24 no stabilizer was added (control experiment). All experiments differed only in the stabilizer used, as is shown in Table 3:

TABLE 3

| Experiment | Stabilizer | Concentration |
|---|---|---|
| 01 | arginine | 10 mmol/l |
| 02 | betaine | 5 mmol/l |
| 03 | dextran | 7.5 mmol/l |
| 04 | desoxycholic acid | 1 mmol/l |
| 05 | caprylate | 10 mmol/l |
| 06 | acetyltryptophan | 10 mmol/l |
| 07 | caprylate | 5 mmol/l |
| 08 | caprylate | 2.5 mmol/l |
| 09 | caprylate | 1.25 mmol/l |
| 10 | heptanoic acid | 2.1 mmol/l |
| 11 | hexanoic acid | 2 mmol/l |
| 12 | capric acid | 2.2 mmol/l |
| 13 | caprylic acid | 2.7 mmol/l |
| 14 | lauric acid | 2.5 mmol/l |
| 15 | myristic acid | 2.5 mmol/l |
| 16 | palmitic acid | 0.1 mmol/l |
| 17 | stearic acid | 0.1 mmol/l |
| 18 | oleic acid | 0.25 mmol/l |
| 19 | linoleic acid | 0.1 mmol/l |
| 20 | linolenic acid | 0.1 mmol/l |
| 21 | arachidonic acid | 0.1 mmol/l |
| 22 | eicosapentaenoic | 0.1 mmol/l |
| | acid | |
| 23 | docosahexaenic | 0.1 mmol/l |
| | acid | |
| 24 | − | 0 |

In summary, in experiments 01-24 only the stabilizer composition (c1) differed.

Experimental Setup and Measuring Equipment

1) The temperature and pH-values were measured with pH-meters M700C (Mettler Toledo Company, Urdorf, Switzerland), with a pH sensor type InPro 3250.

2) The Hach Model 2100P ISO Portable Turbidimeter (HACH, Dusseldorf, Germany) was used to measure the albumin's turbidity. The general description of the Turbidimeter is explained below.
Turbidity
   Turbidity was used for many years as a surrogate for monitoring the combined quantity of particulate material in a water sample. It has been one of the parameters used to provide a basic assessment of water quality. In the present stabilization test the turbidity measurements were used to define the denaturation of the dialysate. Turbidity can be defined as a decrease in the transparency of suspended and some dissolved substances, which causes incident light to be scattered, reflected and attenuated rather than transmitted in straight lines; the higher the intensity of the scattered or attenuated light, the higher the value of turbidity.
Characteristics
   Turbidity can be expressed in nephelometric turbidity units (NTU). Depending on the method used, the turbidity units as NTU can be defined as the intensity of light at a specified wavelength scattered or attenuated by suspended particles or adsorbed at a method-specified angle, usually 90 degrees, from the path of the incident light compared to a synthetic chemically prepared standard.
   The measurement of turbidity is not directly related to a specific number of particles or to a particle shape. As a result, turbidity has historically been seen as a qualitative measurement. Currently, the NTU unit is used for all turbidity measurements and the reported value does not have any traceability to the instrument technology used. At the very last, the units should be listed to the level of NTU (white light, 90 degree detection only), FNU (Formazin Nephelomeric Unit—860 nm Light with 90-degree detection) or FAU (Formazin Attenuation Unit—the detection angle is 180 degrees of the incident light beam) to the measured unit.
   The turbidity value is a quantitative statement of the qualitative phenomenon of turbidity. The objective of measuring turbidity is to obtain information on the concentration of scattering particles in a medium (concentration of solids). This can be done using one of two methods, which fundamentally different: determination of the light loss of the transmitted beam (scatter coefficient) or determination of the intensity of the light scattered sideways.
   Practical interpretation of the turbidity value is achieved by comparison with a standard suspension, i.e. turbidimeters are calibrated with a reference solution (formazine). An instrument that has been calibrated with formazine will measure any formazine concentration correctly. Regarding other turbid media, one cannot be certain of a direct correlation between turbidity value and solids concentration, because the reading will be affected also by particle size and the refractive index of the particles in relation to the medium.
   Attempts to compare the readings produced by different instruments are admissible only if they have the same characteristics with regard to wavelength of the light, scatter angle, optical configuration, calibration and colour compensation. For continuous measurements in those experiment processes, the measuring technique applied (photometer) is also extremely important because of the need for high stability.
   The ratio optical system includes a LED lamp, a 90° detector to monitor scattered light and a transmitted light detector. The microprocessor calculates the ratio of signals from the 90° and transmitted light detector. This ratio technique corrects for interferences from color and/or light absorbing materials (such as activated carbon) and compensates for fluctuations in lamp intensity, providing long-term calibration stability. The optical design also minimizes stray light, increasing measurement accuracy.
3) The Vitros 250 Chemistry System
   The concentration of albumin and other electrolytes were measured during those experiments using the Vitros 250 Chemistry System (Johnson and Johnson, Neckargemuend, Germany).
   The Vitros 250 Chemistry System is an automated clinical chemistry system used for discrete quantitative measurements of analytic concentrations in human fluid specimens. The Vitros 250 System has a throughput of up to 250 results per hour. Methodologies include colorimetric, potentiometric, immuno-rate, and rate tests using multi-layer Vitros Chemistry Slides.
   The slides are packaged in cartridges specific for each test type. Cartridges contain either 18 or 50 slides. The analyzer uses each slide once and after the slide is used, it is discarded. Prior to the sample processing, the cartridges were loaded, the system was calibrated and the samples were programmed.
   The unique properties of these slides eliminate the need to store, mix, and dispose of liquid reagent chemicals and permit reliable analyses with a very small volume of sample.
   A single test result takes approximately two to eight minutes, depending upon the type of test.

Stabilization Test

As already mentioned, a simulation model for the “neutralization zone” was established in order to evaluate the denaturation of albumin (dialysate) in a method as described in Example 2 and to compare the effect of different protein stabilizers.

FIG. 12 provides a schematical overview over the stabilization test, which comprises the following steps:

Step I): A solution comprising HSA, electrolyte and other desired chemicals as described above (e.g., Table 2) is filled into a canister (33), which is kept in 40° C. This solution represents the dialysis fluid/dialysate solution.

Step II): The dialysis fluid is then filled into smaller canisters (34). To each canister (34) a different stabilizer was added. An alkaline composition (31; for example 3 M sodium hydroxide as described below) was then added to the dialysate canister (34) to simulate the alkaline level of the dialysis machine.

Step III): After variable time, an acidic composition (32; for example 0.5 M hydrochloric acid as described below) was added to the dialysate canister (34) to simulate the acid level of the dialysis machine.

Step IV): the turbidity of samples was then measured with the HACH 2100P portable turbidimeter.

### DETAILED DESCRIPTION

I) An albumin-comprising dialysis fluid was prepared from a 5% human serum albumin (HSA) by mixing the acidic composition (a) and the alkaline composition (b) and the necessary solutions and chemicals as known to the skilled person and described in the literature. The solute—buffer mixtures were prepared to a final HSA concentration of 30 mg/ml (0.0454 mmol/L) and, thereafter, filled into 1 L glass canister (33) and mixed continuously with a magnetic stir for ten minutes to dissolve all chemicals in the dialysate. The concentration of albumin was measured before the beginning of experiments using the Vitros 250 Chemistry System. Then the dialysate was separated in 10 small glasses (34), for each sample in 80 ml (same experiment to determine the denaturation time of the dialysate).

The samples were then placed in the water bath in twenty minutes. This was utilized to maintain the dialysate temperature in the range of 40±0.3 ° C. For the monitoring and controlling of dialysate pH and temperature, a pH electrode with an integrated temperature sensor was inserted into the dialysate canister.

II) When the samples reached the desired temperature of 40° C., 3M sodium hydroxide (31) was added to the dialysate to achieve the desired pH of 11.6; the amount of the added alkali was recorded.

After variable mixing time (5, 10, 15 min etc.) with akali, 0.5 M hydrochloric acid (32) was added to the dialysate to achieve pH 3; the amount of the added acid was also recorded.

IV) The concentrations of Na, Cl, Ca, Mg and total protein in the samples (34) were then determined using the Vitros 250 Chemistry System. The results of concentration were then compared to the normal physiological range. The turbidity of the samples was then measured with the HACH 2100P portable turbidimeter. From the measured turbidity, the degree of denaturation of HSA was deduced. The purpose of the stabilization test was to delay the time of denaturation after the alkali was added.

Results:

In control experiment 24, without adding additional stabilizers, albumin denatured in the dialysis fluid within 9.9 min±1.3 min. Without addition of a stabilizer, the time to reach an increase of 50% in turbidity was 20.3±1.9 min.

Addition of arginine, betaine, dextran, sorbitol, gluconate, sulfate, or of any of the fatty acids heptanoic acid, hexanoic acid, capric acid, caprylic acid, lauric acid, myristic acid, palmitic acid, stearic acid, oleic acid, linoleic acid, linolenic acid, arachidonic acid, eicosapentaenoic acid or docosahexaenic acid resulted in an improvement (i.e. prolongation) of the denaturation time in the range of 11.2-22.5% as compared to a dialysis fluid without addition of a stabilizer.

Desoxycholic acid pronouncedly increased the denaturation time to 27±2.1 min. Desoxycholic acid is a naturally occurring substance and is transferred from the blood to the albumin-containing dialysis fluid.

The addition of caplylate 10 mmol/l, 5 mmol/l, 2.5 mmol/l and 1.25 mmol/l resulted in an even more pronounced improvement (i.e. prolongation) of the denaturation time as compared to a dialysis fluid without addition of a stabilizer or with the addition of the stabilizers mentioned above. Namely, caprylate increased the denaturation time to 30.56±6.07 min.

### Example 7

### Effect of Different Protein Stabilizers on the Functionality of Albumin

In addition to the above Example assessing the influence of various stabilizers on the stability of albumin in the dialysis fluid (denaturation time), the present Example addresses the effect of different stabilizers on the functionality of albumin. To this end, the method described in Example 2 (for bilirubin removal see Example 3, FIG. 2A) and the dialysis fluid, the acidic composition (a) and the alkaline composition (b) as described in Example 6 were used. The bilirubin concentration in blood was 510 μmol/l and the porcine blood was treated for one hour in the LK2001 (Hepa Wash GmbH, Munich, Germany).

In the present Example, it was tested whether any of the stabilizers would show an additional effect on bilirubin removal when added to the albumin-containing dialysis fluid. To this end, each of the stabilizers shown in table 4 was separately tested in the present experiment. The different concentrations of the stabilizers in the dialysate are shown in table 4.

Table 4 below shows the results (bilirubin elimination from the blood in %). For the control experiment all stabilizers were removed from the albumin solution and no additional stabilizers were added during the treatment.

TABLE 4

| | | Bilirubin |
|---|---|---|
| Compound | Concentration | elimination in % |
| control | − | 30 |
| arginine | 10 mmol/l | 35 |
| betaine | 5 mmol/l | 32 |
| dextran | 7.5 mmol/l | 32 |
| desoxycholic acid | 1 mmol/l | 40 |
| caprylate | 10 mmol/l | 84 |
| acetyltryptophan | 10 mmol/l | 79 |
| caprylate | 5 mmol/l | 78 |
| caprylate | 2.5 mmol/l | 71 |
| caprylate | 1.25 mmol/l | 62 |
| heptanoic acid | 2.1 mmol/l | 38 |
| hexanoic acid | 2 mmol/l | 36 |
| capric acid | 2.2 mmol/l | 37 |
| caprylic acid | 2.7 mmol/l | 40 |
| lauric acid | 2.5 mmol/l | 35 |
| myristic acid | 2.5 mmol/l | 35 |
| palmitic acid | 0.1 mmol/l | 31 |
| stearic acid | 0.1 mmol/l | 31 |
| oleic acid | 0.25 mmol/l | 51 |
| linoleic acid | 0.1 mmol/l | 32 |
| linolenic acid | 0.1 mmol/l | 33 |
| arachidonic acid | 0.1 mmol/l | 31 |
| eicosapentaenoic acid | 0.1 mmol/l | 33 |
| docosahexaenic acid | 0.1 mmol/l | 32 |

Accordingly, the best results were achieved with caprylate at all concentrations tested and with acetyltryptophan. However, tryptophan and acetyltryptophan are not stable in solution. All tested fatty acids improve the detoxification of bilirubin. being better than the other classes of stabilizers the maximum effect was a 84% reduction by addition of caprylate with a concentration of 10 mmol/l.

### Example 8

### Influence of Different Concentrations of Caprylate on the Stability of the Carrier Protein

To assess the ability of kits according to the present invention comprising different concentrations of caprylate on the stability of the carrier protein such as albumin, the removal of bilirubin from blood was tested using kits according to the present invention comprising different concentrations of caprylate. Experiments were performed using porcine blood and the dialysis device LK2001 (Hepa Wash GmbH, Munich, Germany). In this experiment, kits comprising the following compositions were used:

The acidic composition (a) comprising a biologically compatible acid is an aqueous solution with the following components:

| HCl | 164.0 mmol/l |
|---|---|
| NaCl | 12.0 mmol/l |
| KCl | 7.6 mmol/l |
| Na₂HPO₄ 2H₂O | 1.0 mmol/l |
| MgCl₂ 6H₂O | 1.0 mmol/l |
| CaCl₂ 2H₂O | 2.8 mmol/l |
| pH | 1.05 |

The alkaline composition (b) comprising a biologically compatible base is an aqueous solution with the following components:

| NaOH | 160.0 mmol/l |
|---|---|
| Na₂CO₃ | 54.0 mmol/l |
| pH | 12.6 |

The acidic composition (a) and the alkaline composition (b) of the kits were used to directly treat the carrier protein-containing multiple pass dialysis fluid.

In addition to the acidic composition (a) and to the alkaline composition (b), a stabilizer/nutrient composition (c5) with the following components:

| | |
|---|---|
| Na-caprylate (C₈H₁₅O₂Na) | 0-240 mmol/l |
| Glucose | 40 w/w % |

Identical acidic compositions (a) and alkaline compositions (b) were used in all kits. The kits differed only in the concentration of Na-caprylate (C₈H₁₅O₂Na). Table 5 shows the Na-caprylate (C₈H₁₅O₂Na) concentrations used.

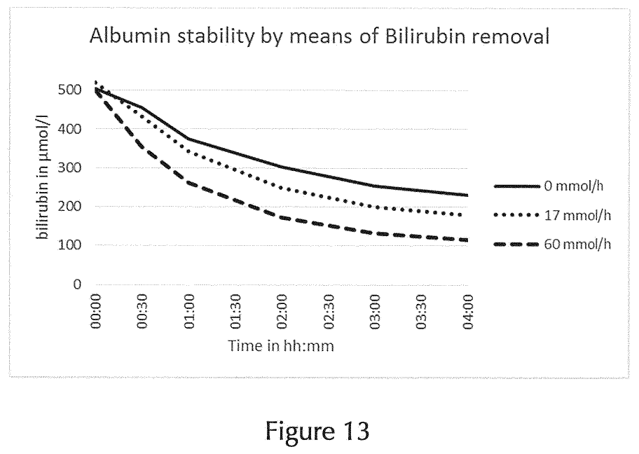

TABLE 5

| Kit/ | Na-caprylate | Referred to in |
|---|---|---|
| experiment | concentration | Fig. 13 as |
| 8A | 0 (control) | 0 mmol/h |
| 8B | 240 mmol/l | 17 mmol/h |
| 8C | 240 mmol/l | 60 mmol/h |

For each kit/experiment, porcine blood was treated for 4 h in the dialysis device LK2001 (Hepa Wash GmbH, Munich, Germany) as described in Example 2 and the concentration of bilirubin in the blood was measured.

Results are shown in FIG. 13. As can be retrieved from FIG. 13, the higher the concentration of caprylate added to the dialysate, the more bilirubin is removed. These results indicate that the stability of albumin increases with higher concentrations of caprylate.

### Example 9

### Stability of Glucose in Solutions with or without Caprylate

To assess the influence of a protein stabilizer, such as caprylate, on the stability of a sugar, such as glucose, when present in the same composition, HMF (5-(Hydroxymethyl)-2-furaldehyd) levels were assessed. HMF is an organic compound derived from dehydration of certain sugars. Accordingly, the HMF level is indicative for the stability of sugars with the more HMF the less stable the sugar.

To this end, a stabilizer/nutrient composition (c5) comprising 428 mmol/l C₈H₁₅NaO₂ and 2220 mmol/l D-glucose and a nutrient composition (c2) comprising 2220 mmol/l D-glucose, but no caprylate, were exposed to different temperatures and the HMF levels were assessed.

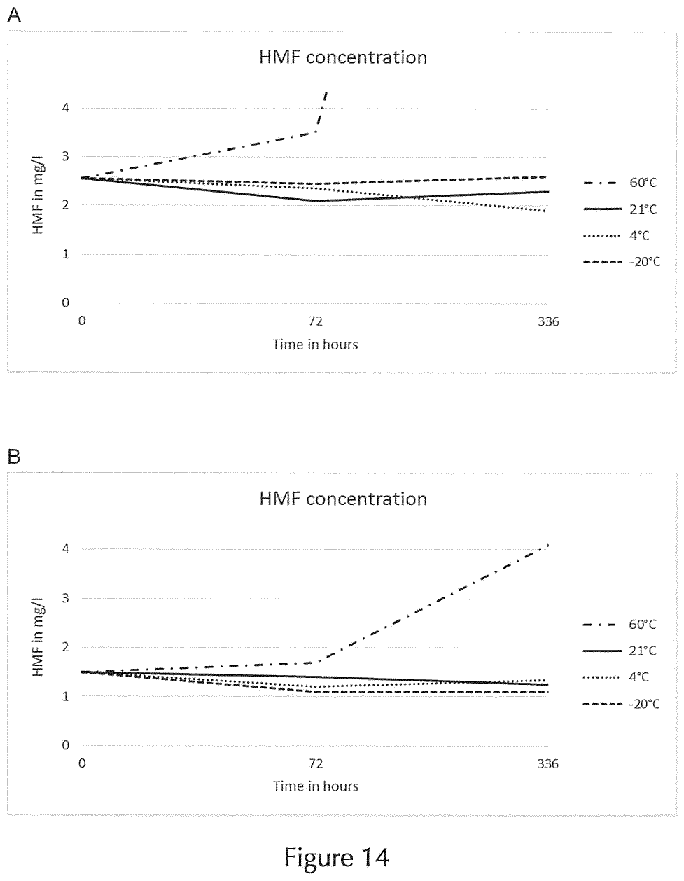

Results are shown in FIG. 14. As can be retrieved from FIG. 14, D-glucose in a stabilizer/nutrient composition (c5), which comprises for example caprylate, (FIG. 14B) is more stable then D-glucose alone in a nutrient composition (c2) (FIG. 14A). 5-(Hydroxymethyl)-2-furaldehyd (HMF) is a dehydration product of D-Fructose. Therefore, the higher the concentration of HMF the less stable the glucose in the composition. In general, FIG. 14 shows that higher temperatures lead to an increase in HMF concentration. The composition without stabilizer shown in FIG. 14A shows at all storage temperatures considerably more HMF as compared to the composition with stabilizer shown in FIG. 14B. Therefore, the addition of a stabilizer to the composition increases the stability of glucose.

1. A kit for treating a carrier protein-containing multiple pass dialysis fluid comprising
(a) an acidic composition comprising a biologically compatible acid, and
(b) an alkaline composition comprising a biologically compatible base, wherein the ratio of the concentration of the biologically compatible acid in the acidic composition (a) to the concentration of the biologically compatible base in the alkaline composition (b) is in the range from 0.7 to 1.3, preferably in the range from 0.75 to 1.25 and more preferably in the range from 0.8 to 1.2 and wherein the concentration of the biologically compatible acid in the acidic composition and the concentration of the biologically compatible base in the alkaline composition is at least 50 mmol/l and no more than 500 mmol/l.

2. The kit according to claim 1, wherein the concentration of the biologically compatible acid in the acidic composition (a) and the concentration of the biologically compatible base in the alkaline composition (b) is at least 60 mmol and no more than 400 mmol/l, preferably at least 70 mmol/l and no more than 300 mmol/l and more preferably at least 100 mmol/l and no more than 200 mmol/l.

3. The kit according to claim 1, wherein the acidic composition (a) is an aqueous solution of the biologically compatible acid, optionally comprising further components, and wherein the alkaline composition (b) is an aqueous solution of the biologically compatible base, optionally comprising further components.

4. The kit according to claim 1, wherein the kit comprises a stabilizer for a carrier protein, in particular a stabilizer for albumin.

5. The kit according to claim 4, wherein the kit comprises
(c1) a stabilizer composition comprising the stabilizer for a carrier protein, in particular the stabilizer for albumin,
wherein the stabilizer composition (c1) is different from the acidic composition (a) and from the alkaline composition (b).

6. The kit according to claim 4, wherein the stabilizer for a carrier protein, in particular the stabilizer for albumin, is selected from the group consisting of amino acids, salts of amino acids, derivatives of amino acids, fatty acids, salts of fatty acids, derivatives of fatty acids, sugars, polyols and osmolytes.

7. The kit according to claim 6, wherein the stabilizer is selected from the group consisting of fatty acids, salts of fatty acids and derivatives of fatty acids.

8. The kit according to claim 7, wherein the stabilizer is selected from the group consisting of caprylate, caprylic acid, caprate, capric acid, caproic acid and caproate, preferably the stabilizer is a caprylate.

9. The kit according to claim 5, wherein the concentration of the stabilizer in the stabilizer composition (c1) is in the range from 1 to 2500 mmol/l, more preferably from 50 to 1500 mmol/l, even more preferably from 100 to 1000 mmol/l and most preferably from 150 to 500 mmol/l.

10. The kit according to claim 1, wherein the kit further comprises (c2) a nutrient composition comprising a nutrient, in particular a sugar, wherein the nutrient composition (c2) is different from the acidic composition (a) and from the alkaline composition (b).

11. The kit according to claim 10, wherein the nutrient is glucose.

12. The kit according to claim 1, wherein the kit comprises at least one component selected from the group consisting of sodium, chloride, calcium, magnesium, potassium, phosphate and carbonate/bicarbonate.

13. The kit according to claim 12, wherein at least one component selected from the group consisting of sodium, chloride, calcium, magnesium, potassium and phosphate is comprised by the acidic composition (a).

14. The kit according to claim 12, wherein at least one component selected from the group consisting of sodium, chloride, potassium, phosphate, carbonate/bicarbonate and Tris is comprised by the alkaline composition (b).

15. The kit according to claim 12, wherein the kit further comprises (c3) an electrolyte composition comprising at least one component selected from the group consisting of sodium, chloride, calcium, magnesium, potassium and phosphate, wherein the electrolyte composition (c3) is different from the acidic composition (a) and from the alkaline composition (b).

16. The kit according to claim 12, wherein the source of sodium is NaOH, Na₂CO₃, Na₂HPO₄, NaHCO₃, NaCl, and/or a sodium salt of lactate, acetate, gluconate, citrate, maleate, tartrate and/or of fatty acids such as caprylate.

17. The kit according to claim 12, wherein the source of chloride is HCl, NaCl, KCl, MgCl₂, and/or CaCl₂.

18. The kit according to claim 12, wherein the source of potassium is KOH and/or KCl.

19. The kit according to claim 12, wherein the source of calcium is CaCl₂, CaCO₃, and/or a calcium salt of lactate, acetate, gluconate, citrate, maleate, tartrate and/or of fatty acids, preferably the source of calcium is a calcium salt of lactate, acetate, gluconate, citrate, maleate and/or tartrate.

20. The kit according to claim 12, wherein the source of magnesium is MgCl₂, MgCO₃, and/or a magnesium salt of lactate, acetate, gluconate, citrate, maleate, tartrate and/or of fatty acids, preferably the source of magnesium is a magnesium salt of lactate, acetate, gluconate, citrate, maleate and/or tartrate.

21. The kit according to claim 12, wherein the kit further comprises (c4) a buffering composition comprising a buffering agent, in particular carbonate/bicarbonate, wherein the buffering composition (c4) is different from the acidic composition (a) and from the alkaline composition (b).

22. The kit according to claim 5, wherein the kit comprises a stabilizer composition (c1) and a nutrient composition (c2) and wherein the stabilizer composition (c1) and the nutrient composition (c2) are the same composition (c5) or different compositions.

23. The kit according to claim 15, wherein the kit comprises an electrolyte composition (c3) and a buffering composition (c4) and wherein the electrolyte composition (c3) and the buffering composition (c4) are the same composition (c6) or different compositions.

24. The kit according to claim 5, wherein the kit comprises a stabilizer composition (c1) and an electrolyte composition (c3) and wherein the stabilizer composition (c1) and the electrolyte composition (c3) are the same composition (c7) or different compositions.

25. The kit according to claim 10, wherein the kit comprises a nutrient composition (c2) and an electrolyte composition (c3) and wherein the nutrient composition (c2) and the electrolyte composition (c3) are the same composition (c8) or different compositions.

26. The kit according to claim 5, wherein the kit comprises a stabilizer composition (c1) and a buffering composition (c4) and wherein the stabilizer composition (c1) and the buffering composition (c4) are the same composition (c9) or different compositions.

27. The kit according to claim 10, wherein the kit comprises a nutrient composition (c2) and a buffering composition (c4) and wherein the nutrient composition (c2) and the buffering composition (c4) are the same composition (c10) or different compositions.

28. The kit according to claim 5, wherein the kit comprises a stabilizer composition (c1), a nutrient composition (c2) and an electrolyte composition (c3) and wherein the stabilizer composition (c1), the nutrient composition (c2) and the electrolyte composition (c3) are the same composition (c11) or different compositions.

29. The kit according to claim 5, wherein the kit comprises a stabilizer composition (c1), a nutrient composition (c2), an electrolyte composition (c3) and a buffering composition (c4) and wherein the stabilizer composition (c1), the nutrient composition (c2), the electrolyte composition (c3) and the buffering composition (c4) are the same composition (c12) or different compositions.

30. The kit according to claim 1, wherein
(a) the acidic composition (a) comprises at least one component selected from the group consisting of sodium, chloride, calcium, magnesium, potassium and phosphate; and
(b) the alkaline composition (b) comprises at least one component selected from the group consisting of sodium, chloride, potassium, phosphate and carbonate/bicarbonate and, optionally, a stabilizer for a carrier protein, in particular a stabilizer for albumin.

31. The kit according to claim 1, wherein
(a) the acidic composition (a) comprises at least one component selected from the group consisting of sodium, chloride, calcium, magnesium, potassium and phosphate; and
(b) the alkaline composition (b) comprises at least one component selected from the group consisting of sodium, chloride, potassium, phosphate and carbonate/bicarbonate; and
wherein the kit further comprises
a stabilizer composition (c1) comprising a stabilizer for a carrier protein, in particular a stabilizer for albumin, wherein the stabilizer composition (c1) is different from the acidic composition (a) and from the alkaline composition (b); and/or
a nutrient composition (c2) comprising a nutrient, in particular a sugar, wherein the nutrient composition (c2) is different from the acidic composition (a) and from the alkaline composition (b).

32. The kit according to claim 31, wherein the kit comprises a stabilizer/nutrient composition (c5), which comprises
a sugar, preferably glucose, and
a stabilizer for a carrier protein, in particular a stabilizer for albumin, preferably a caprylate,
wherein the composition (c5) is different from the acidic composition (a) and from the alkaline composition (b).

33. The kit according to claim 32, wherein the kit comprises a stabilizer/nutrient/electrolyte composition (c11), which comprises
a sugar, preferably glucose,
a stabilizer for a carrier protein, in particular a stabilizer for albumin, preferably caprylate, and
at least one component selected from the group consisting of sodium, chloride, calcium, magnesium, potassium and phosphate; and
wherein the composition (c11) is different from the acidic composition (a) and from the alkaline composition (b).

34. Use of a kit according to claim 1 for treating, in particular regenerating, a carrier protein-containing multiple pass dialysis fluid.

35. A method for regenerating a carrier protein-containing multiple pass dialysis fluid, wherein the carrier protein-containing multiple pass dialysis fluid is treated, in particular regenerated,
with an acidic composition (a), which comprises a biologically compatible acid, and
with an alkaline composition (b), which comprises a biologically compatible base,
wherein the ratio of the concentration of the biologically compatible acid in the acidic composition (a) to the concentration of the biologically compatible base in the alkaline composition (b) is in the range from 0.7 to 1.3, preferably in the range from 0.75 to 1.25 and more preferably in the range from 0.8 to 1.2 and wherein the concentration of the biologically compatible acid in the acidic composition and the concentration of the biologically compatible base in the alkaline composition is at least 50 mmol/l and no more than 500 mmol/l.

36. The method according to claim 35, wherein the treatment of the carrier protein-containing multiple pass dialysis fluid with the acidic composition (a) and with the alkaline composition (b) occurs consecutively.

37. The method according to claim 35 comprising the following steps:
(i) passing the carrier protein-containing multiple pass dialysis fluid through a dialyzer,
(ii) dividing the flow of the carrier protein-containing multiple pass dialysis fluid into a first flow and a second flow,
(iii) adding the acidic composition (a) to the first flow of the carrier protein-containing multiple pass dialysis fluid and the alkaline composition (b) to the second flow of the carrier protein-containing multiple pass dialysis fluid,
(iv) filtration of the first flow of the carrier protein-containing multiple pass dialysis fluid treated with the acidic composition (a) and of the second flow of the carrier protein-containing multiple pass dialysis fluid treated with the alkaline composition (b),
(v) rejoining the first flow of the carrier protein-containing multiple pass dialysis fluid treated with the acidic composition (a) and the second flow of the carrier protein-containing multiple pass dialysis fluid treated with the alkaline composition (b), and
(vi) optionally, performing a further cycle beginning with step (i).

38. The method according to claim 37, wherein in step (iii) the addition of the acidic composition (a) to the first flow of the carrier protein-containing multiple pass dialysis fluid occurs at about the same time as the addition of the alkaline composition (b) to the second flow of the carrier protein-containing multiple pass dialysis fluid.

39. The method according to claim 35, wherein the carrier protein-containing multiple pass dialysis fluid is treated with a stabilizer composition (c1), which comprises a stabilizer for a carrier protein, in particular a stabilizer for albumin, and which is different from the acidic composition (a) and from the alkaline composition (b).

40. The method according to claim 35, wherein the carrier protein-containing multiple pass dialysis fluid is treated with a nutrient composition (c2), which comprises a nutrient, in particular a sugar, and which is different from the acidic composition (a) and from the alkaline composition (b).

41. The method Method according to claim 35, wherein the carrier protein-containing multiple pass dialysis fluid is treated with an electrolyte composition (c3), which comprises at least one component selected from the group consisting of sodium, chloride, calcium, magnesium, potassium and phosphate and which is different from the acidic composition (a) and from the alkaline composition (b).

42. The method according to claim 35, wherein the carrier protein-containing multiple pass dialysis fluid is treated with a buffering composition (c4), which comprises a buffering agent, in particular carbonate/bicarbonate, and which is different from the acidic composition (a) and from the alkaline composition (b).

43. The method according to claim 35, wherein the carrier protein-containing multiple pass dialysis fluid is treated with a stabilizer composition (c1), a nutrient composition (c2) and/or an electrolyte composition (c3) and wherein the stabilizer composition (c1), the nutrient composition (c2) and/or the electrolyte composition (c3) are the same composition or different compositions.

44. The method according to claim 35, wherein the carrier protein-containing multiple pass dialysis fluid is treated with a stabilizer composition (c1), a nutrient composition (c2), an electrolyte composition (c3) and/or buffering composition (c4) and wherein the stabilizer composition (c1), the nutrient composition (c2), the electrolyte composition (c3) and/or the buffering composition (c4) are the same composition or different compositions.

45. The method according to claim 44, wherein the stabilizer composition (c1), the nutrient composition (c2), the electrolyte composition (c3) and/or the buffering composition (c4) are added to the carrier protein-containing multiple pass dialysis fluid
after the treatment of the carrier protein-containing multiple pass dialysis fluid with the acidic composition (a) and with the alkaline composition (b), preferably after step (v) of claim 37, and
before passing the carrier protein-containing multiple pass dialysis fluid through the dialyzer.

46. The method according to claim 37 comprising a step (v-1) following upon step (v) and preceding step (vi):
(v-1) adding to the carrier protein-containing multiple pass dialysis fluid: (i) a stabilizer composition (c1), which comprises a stabilizer for a carrier protein, in particular a stabilizer for albumin; (ii) a nutrient composition (c2), which comprises a nutrient, in particular a sugar; (iii) an electrolyte composition (c3), which comprises at least one component selected from the group consisting of sodium, chloride, calcium, magnesium, potassium and phosphate and/or (iv) a buffering composition (c4), which comprises a buffering agent, in particular carbonate/bicarbonate;
wherein the stabilizer composition (c1), the nutrient composition (c2), the electrolyte composition (c3) and/or the buffering composition (c4) are the same composition or different compositions.

47. A method for providing a carrier protein-containing multiple pass dialysis fluid comprising the following steps:
(i) providing an acidic composition (a), which comprises a biologically compatible acid, and an alkaline composition (b), which comprises a biologically compatible base,
wherein the ratio of the concentration of the biologically compatible acid in the acidic composition (a) to the concentration of the biologically compatible base in the alkaline composition (b) is in the range from 0.7 to 1.3, preferably in the range from 0.75 to 1.25 and more preferably in the range from 0.8 to 1.2 and wherein the concentration of the biologically compatible acid in the acidic composition (a) and the concentration of the biologically compatible base in the alkaline composition (b) is at least 50 mmol/l and no more than 500 mmol/l,
(ii) merging the acidic composition (a) with the alkaline composition (b), and
(iii) adding a carrier protein, preferably albumin, more preferably human serum albumin (HSA).

48. The method according to claim 47 comprising a step (ii-1) following upon step (ii) and preceding step (iii):
(ii-1) adding (i) a stabilizer composition (c1), which comprises a stabilizer for a carrier protein, in particular a stabilizer for albumin; (ii) a nutrient composition (c2), which comprises a sugar; (iii) an electrolyte composition (c3), which comprises at least one component selected from the group consisting of sodium, chloride, calcium, magnesium, potassium and phosphate and/or (iv) a buffering composition (c4), which comprises a buffering agent, in particular carbonate/bicarbonate,
wherein the stabilizer composition (c1), the nutrient composition (c2), the electrolyte composition (c3) and/or the buffering composition (c4) are the same composition or different compositions.

49. Use of a kit according to claim 1 in a method according to claim 35.
